SEQUENCE LISTING (9 sequences)

SEQ ID NO: 1 - protein, 6 aa, Rattus rattus
ESESRG

SEQ ID NO: 2 - protein, 6 aa, Rattus sp.
ESENVY

SEQ ID NO: 3 - protein, 883 aa, Rattus sp.
MIPLLLSLLAALVLTQAPAALADDLKEDSSEDRAFRVRIGAAQLRGVLGGALAIPCHVHHLRPPPSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACYGDMDGYPGVRNYGVVGPDDLYDVYCYAEDLNGELFLGAPPGKLTWEEARDYCLERGAQIASTGQLYAAWNGGLDRCSPGWLADGSVRYPIITPSQRCGGGLPGVKTLFLFPNQTGFPSKQNRFNVYCFRDSAHPSAFSEASSPASDGLEAIVTVTEKLEELQLPQEAVESENVYAIYSIPITEDGGGGSSTPEDPAEAPRTPLESETQSVAPPTGSSEEEGEALEEEERFKDTETPKEEKEQENLWVWPTELSSPLPTGLETEHSLSQVSPPAQAVLQLGASPSPRPPRVHGPPAETLQPPREGSLTSTPDGAREVAGETGSPELSGVPRESEEAGSSSLEDGPSLLPATWAPVGTRELETPSEEKSGRTVLTGTSVQAQPVLPTDSASRGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLAYAVDTVLRYRCRDGLAQRNLPLIRCQENGLWEAPQISCVPRRPARALRSMTAPEGPRGQLPRQRKALLTPPSSL

SEQ ID NO: 4 - DNA, 2652 bp, Rattus sp.
atgatcccattgcttctgtccctgctggcagctctggtcctgacccaagcccctgcagccctcgctgatgacctgaaagaagacagctcagaggatcgagcctttcgggtgcgcatcggtgccgcgcagctgcggggtgtgctgggcggtgccctggccatcccatgccacgtccaccacctgaggccgccgcccagccgccgggccgcgccgggctttccccgagtcaaatggaccttcctgtccggggaccgggaggtggaggtgctggtggcgcgcgggctgcgcgtcaaggtaaacgaagcctatcggttccgcgtggcgctgcctgcctaccccgcatcgctcacagatgtgtctttagtattgagcgaactgcggcccaatgattccggggtctatcgctgcgaggtccagcacggtatcgacgacagcagtgatgctgtggaagtcaaggtcaaaggggtcgtcttcctctaccgagagggctctgcccgctatgctttctccttcgctggagcccaggaagcctgtgctcgcatcggagcccgaattgccacccctgagcagctgtatgctgcctacctcggcggctatgaacagtgtgatgctggctggctgtccgaccaaaccgtgaggtaccccatccagaacccacgagaagcctgttatggagacatggatggctaccctggagtgcggaattacggagtggtgggtcctgatgatctctacgatgtctactgttatgccgaagacctaaatggagaactgttcctaggtgcccctcccggcaagctgacgtgggaggaggctcgggactactgtctggaacgcggtgctcagatcgctagcacgggccagctatacgcggcatggaatggcggcttggacagatgtagccctggctggctggctgatggcagtgtgcggtaccccatcatcacgcccagccaacgctgtgggggaggcctgccaggagtcaagaccctcttcctctttcccaaccagactggcttccccagcaagcagaaccgcttcaatgtctactgcttccgagactctgcccatccctctgccttctctgaggcctccagcccagcctctgatggactagaggccattgtcacagtgacagagaagctggaggaactgcagttgcctcaggaagctgtggagagcgagaatgtttacgcgatctactccatccccatcacagaagatgggggaggaggaagctctaccccagaagacccagcagaggcccccaggactcctctagaatcagaaacccaatccgttgcaccacctaccgggtcctcagaagaggaaggcgaagccctggaggaagaagaaagattcaaagacacagagactccgaaggaagagaaggagcaggagaacctgtgggtgtggcccacggagctcagcagccctctccctactggcttggaaacagagcactcactctcccaggtgtccccaccagcccaggcagttctacagctgggtgcatcaccttctcccaggcctccaagggtccatggaccgcctgcagagactttgcaacccccaagggagggaagcctcacatctactccagatggggcaagagaagtagcgggggaaactgggagccctgagctctctggggttcctcgagaaagcgaggaggcaggaagctccagcttggaggatggcccttccctccttccagcgacatgggcccctgtgggtaccagggagctggagaccccctcagaagagaagtctggaagaactgttctgacaggcacatcagtgcaggcccagccagtgctgcccaccgacagtgccagccgaggtggagtggctgtggctccctcatcaggtgactgtatccccagcccctgccacaatggtgggacatgcttggaggagaaggagggtttccgctgcctctgtttgccaggctatgggggggacctgtgcgatgttggcctccacttctgcagcccgggctgggaggccttccagggtgcctgctacaagcacttttccacacgaaggagttgggaggaggcagaaagccagtgccgagcgctaggggctcatctgaccagcatctgcacccctgaggagcaggactttgtcaacgatcgatacagggagtaccagtggattgggctcaatgacaggaccatcgagggtgacttcctgtggtcagatggtgcccctctgctctatgaaaactggaaccctgggcagcctgacagctacttcctgtctggggagaactgtgtggtcatggtgtggcatgaccagggacagtggagtgatgtaccctgcaactaccacctatcctacacctgcaagatggggcttgtgtcatgtggacctccaccacagctgcccctggctcaaatatttggtcgccctcggctggcctacgcggtggacactgtgcttcgatatcggtgccgagacgggctggcccagcgcaacttgccgttgatccgctgccaggagaatgggctttgggaggcccctcagatttcttgcgtgccccgaagacctgcccgtgctctccgctcaatgaccgccccagaaggaccacggggacagctcccgaggcagaggaaagcactgttgacacctccctccagtctctag

SEQ ID NO: 5 - DNA, 2652 bp, Rattus rattus
atgatcccattgcttctgtccctgctggcagctctggtcctgacccaagcccctgcagccctcgctgatgacctgaaagaagacagctcagaggatcgagcctttcgggtgcgcatcggtgccgcgcagctgcggggtgtgctgggcggtgccctggccatcccatgccacgtccaccacctgaggccgccgcccagccgccgggccgcgccgggctttccccgagtcaaatggaccttcctgtccggggaccgggaggtggaggtgctggtggcgcgcgggctgcgcgtcaaggtaaacgaagcctatcggttccgcgtggcgctgcctgcctaccccgcatcgctcacagatgtgtctttagtattgagcgaactgcggcccaatgattccggggtctatcgctgcgaggtccagcacggtatcgacgacagcagtgatgctgtggaagtcaaggtcaaaggggtcgtcttcctctaccgagagggctctgcccgctatgctttctccttcgctggagcccaggaagcctgtgctcgcatcggagcccgaattgccacccctgagcagctgtatgctgcctacctcggcggctatgaacagtgtgatgctggctggctgtccgaccaaaccgtgaggtaccccatccagaacccacgagaagcctgttatggagacatggatggctaccctggagtgcggaattacggagtggtgggtcctgatgatctctacgatgtctactgttatgccgaagacctaaatggagaactgttcctaggtgcccctcccggcaagctgacgtgggaggaggctcgggactactgtctggaacgcggtgctcagatcgctagcacgggccagctatacgcggcatggaatggcggcttggacagatgtagccctggctggctggctgatggcagtgtgcggtaccccatcatcacgcccagccaacgctgtgggggaggcctgccaggagtcaagaccctcttcctctttcccaaccagactggcttccccagcaagcagaaccgcttcaatgtctactgcttccgagactctgcccatccctctgccttctctgaggcctccagcccagcctctgatggactagaggccattgtcacagtgacagagaagctggaggaactgcagttgcctcaggaagctgtggagagcgagtctcgtggggcgatctactccatccccatcacagaagatgggggaggaggaagctctaccccagaagacccagcagaggcccccaggactcctctagaatcagaaacccaatccgttgcaccacctaccgggtcctcagaagaggaaggcgaagccctggaggaagaagaaagattcaaagacacagagactccgaaggaagagaaggagcaggagaacctgtgggtgtggcccacggagctcagcagccctctccctactggcttggaaacagagcactcactctcccaggtgtccccaccagcccaggcagttctacagctgggtgcatcaccttctcccaggcctccaagggtccatggaccgcctgcagagactttgcaacccccaagggagggaagcctcacatctactccagatggggcaagagaagtagcgggggaaactgggagccctgagctctctggggttcctcgagaaagcgaggaggcaggaagctccagcttggaggatggcccttccctccttccagcgacatgggcccctgtgggtaccagggagctggagaccccctcagaagagaagtctggaagaactgttctgacaggcacatcagtgcaggcccagccagtgctgcccaccgacagtgccagccgaggtggagtggctgtggctccctcatcaggtgactgtatccccagcccctgccacaatggtgggacatgcttggaggagaaggagggtttccgctgcctctgtttgccaggctatgggggggacctgtgcgatgttggcctccacttctgcagcccgggctgggaggccttccagggtgcctgctacaagcacttttccacacgaaggagttgggaggaggcagaaagccagtgccgagcgctaggggctcatctgaccagcatctgcacccctgaggagcaggactttgtcaacgatcgatacagggagtaccagtggattgggctcaatgacaggaccatcgagggtgacttcctgtggtcagatggtgcccctctgctctatgaaaactggaaccctgggcagcctgacagctacttcctgtctggggagaactgtgtggtcatggtgtggcatgaccagggacagtggagtgatgtaccctgcaactaccacctatcctacacctgcaagatggggcttgtgtcatgtggacctccaccacagctgcccctggctcaaatatttggtcgccctcggctggcctacgcggtggacactgtgcttcgatatcggtgccgagacgggctggcccagcgcaacttgccgttgatccgctgccaggagaatgggctttgggaggcccctcagatttcttgcgtgccccgaagacctgcccgtgctctccgctcaatgaccgccccagaaggaccacggggacagctcccgaggcagaggaaagcactgttgacacctccctccagtctctag

SEQ ID NO: 6 - protein, 883 aa, Rattus rattus
MIPLLLSLLAALVLTQAPAALADDLKEDSSEDRAFRVRIGAAQLRGVLGGALAIPCHVHHLRPPPSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACYGDMDGYPGVRNYGVVGPDDLYDVYCYAEDLNGELFLGAPPGKLTWEEARDYCLERGAQIASTGQLYAAWNGGLDRCSPGWLADGSVRYPIITPSQRCGGGLPGVKTLFLFPNQTGFPSKQNRFNVYCFRDSAHPSAFSEASSPASDGLEAIVTVTEKLEELQLPQEAVESESRGAIYSIPITEDGGGGSSTPEDPAEAPRTPLESETQSVAPPTGSSEEEGEALEEEERFKDTETPKEEKEQENLWVWPTELSSPLPTGLETEHSLSQVSPPAQAVLQLGASPSPRPPRVHGPPAETLQPPREGSLTSTPDGAREVAGETGSPELSGVPRESEEAGSSSLEDGPSLLPATWAPVGTRELETPSEEKSGRTVLTGTSVQAQPVLPTDSASRGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLAYAVDTVLRYRCRDGLAQRNLPLIRCQENGLWEAPQISCVPRRPARALRSMTAPEGPRGQLPRQRKALLTPPSSL

SEQ ID NO: 7 - DNA, 2878 bp, Homo sapiens
tgtggcactgcctgcgtacccaaccccagccctgggtagcctgcagcatggcccagctgttcctgcccctgctggcagccctggtcctggcccaggctcctgcagctttagcagatgttctggaaggagacagctcagaggaccgcgcttttcgcgtgcgcatcgcgggcgacgcgccactgcagggcgtgctcggcggcgccctcaccatcccttgccacgtccactacctgcggccaccgccgagccgccgggctgtgctgggctctccgcgggtcaagtggactttcctgtcccggggccgggaggcagaggtgctggtggcgcggggagtgcgcgtcaaggtgaacgaggcctaccggttccgcgtggcactgcctgcgtacccagcgtcgctcaccgacgtctccctggcgctgagcgagctgcgccccaacgactcaggtatctatcgctgtgaggtccagcacggcatcgatgacagcagcgacgctgtggaggtcaaggtcaaaggggtcgtctttctctaccgagagggctctgcccgctatgctttctccttttctggggcccaggaggcctgtgcccgcattggagcccacatcgccaccccggagcagctctatgccgcctaccttgggggctatgagcaatgtgatgctggctggctgtcggatcagaccgtgaggtatcccatccagaccccacgagaggcctgttacggagacatggatggcttccccggggtccggaactatggtgtggtggacccggatgacctctatgatgtgtactgttatgctgaagacctaaatggagaattgttcctgggtgaccctccagagaagctgacattggaggaagcacgggcgtactgccaggagcggggtgcagagattgccaccacgggccaactgtatgcagcctgggatggtggcctggaccactgcagcccagggtggctagctgatggcagtgtgcgctaccccatcgtcacacccagccagcgctgtggtgggggcttgcctggtgtcaagactctcttcctcttccccaaccagactggcttccccaataagcacagccgcttcaacgtctactgcttccgagactcggcccagccttctgccatccctgaggcctccaacccagcctccaacccagcctctgatggactagaggctatcgtcacagtgacagagaccctggaggaactgcagctgcctcaggaagccacagagagtgaatcccgtggggccatctactccatccccatcatggaggacggaggaggtggaagctccactccagaagacccagcagaggcccctaggacgctcctagaatttgaaacacaatccatggtaccgcccacggggttctcagaagaggaaggtaaggcattggaggaagaagagaaatatgaagatgaagaagagaaagaggaggaagaagaagaggaggaggtggaggatgaggctctgtgggcatggcccagcgagctcagcagcccgggccctgaggcctctctccccactgagccagcagcccaggaggagtcactctcccaggcgccagcaagggcagtcctgcagcctggtgcatcaccacttcctgatggagagtcagaagcttccaggcctccaagggtccatggaccacctactgagactctgcccactcccagggagaggaacctagcatccccatcaccttccactctggttgaggcaagagaggtgggggaggcaactggtggtcctgagctatctggggtccctcgaggagagagcgaggagacaggaagctccgagggtgccccttccctgcttccagccacacgggcccctgagggtaccagggagctggaggccccctctgaagataattctggaagaactgccccagcagggacctcagtgcaggcccagccagtgctgcccactgacagcgccagccgaggtggagtggccgtggtccccgcatcaggtgactgtgtccccagcccctgccacaatggtgggacatgcttggaggaggaggaaggggtccgctgcctatgtctgcctggctatgggggggacctgtgcgatgttggcctccgcttctgcaaccccggctgggacgccttccagggcgcctgctacaagcacttttccacacgaaggagctgggaggaggcagagacccagtgccggatgtacggcgcgcatctggccagcatcagcacacccgaggaacaggacttcatcaacaaccggtaccgggagtaccagtggatcggactcaacgacaggaccatcgaaggcgacttcttgtggtcggatggcgtccccctgctctatgagaactggaaccctgggcagcctgacagctacttcctgtctggagagaactgcgtggtcatggtgtggcatgatcagggacaatggagtgacgtgccctgcaactaccacctgtcctacacctgcaagatggggctggtgtcctgtgggccgccaccggagctgcccctggctcaagtgttcggccgcccacggctgcgctatgaggtggacactgtgcttcgctaccggtgccgggaaggactggcccagcgcaatctgccgctgatccgatgccaagagaacggtcgttgggaggccccccagatctcctgtgtgcccagaagacctgcccgagctctgcacccagaggaggacccagaaggacgtcaggggaggctactgggacgctggaaggcgctgttgatccccccttccagccccatgccaggtccctagggggcaaggccttgaacactgccggccacagcactgccctgtcacccaaattttccctcacaccctgcgctcaccacaggaagtgacaacatgac

SEQ ID NO: 8 - protein, 911 aa, Homo sapiens
MAQLFLPLLAALVLAQAPAALADVLEGDSSEDRAFRVRIAGDAPLQGVLGGALTIPCHVHYLRPPPSRRAVLGSPRVKWTFLSRGREAEVLVARGVRVKVNEAYRFRVALPAYPASLTDVSLALSELRPNDSGIYRCEVQHGIDDSSDAVEVKVKGVVFLYREGSARYAFSFSGAQEACARIGAHIATPEQLYAAYLGGYEQCDAGWLSDQTVRYPIQTPREACYGDMDGFPGVRNYGVVDPDDLYDVYCYAEDLNGELFLGDPPEKLTLEEARAYCQERGAEIATTGQLYAAWDGGLDHCSPGWLADGSVRYPIVTPSQRCGGGLPGVKTLFLFPNQTGFPNKHSRFNVYCFRDSAQPSAIPEASNPASNPASDGLEAIVTVTETLEELQLPQEATESESRGAIYSIPIMEDGGGGSSTPEDPAEAPRTLLEFETQSMVPPTGFSEEEGKALEEEEKYEDEEEKEEEEEEEEVEDEALWAWPSELSSPGPEASLPTEPAAQEESLSQAPARAVLQPGASPLPDGESEASRPPRVHGPPTETLPTPRERNLASPSPSTLVEAREVGEATGGPELSGVPRGESEETGSSEGAPSLLPATRAPEGTRELEAPSEDNSGRTAPAGTSVQAQPVLPTDSASRGGVAVVPASGDCVPSPCHNGGTCLEEEEGVRCLCLPGYGGDLCDVGLRFCNPGWDAFQGACYKHFSTRRSWEEAETQCRMYGAHLASISTPEEQDFINNRYREYQWIGLNDRTIEGDFLWSDGVPLLYENWNPGQPDSYFLSGENCVVMVWHDQGQWSDVPCNYHLSYTCKMGLVSCGPPPELPLAQVFGRPRLRYEVDTVLRYRCREGLAQRNLPLIRCQENGRWEAPQISCVPRRPARALHPEEDPEGRQGRLLGRWKALLIPPSSPMPGP

SEQ ID NO: 9 - protein, 7 aa, Rattus sp.
QEAVESE

(19) United States
(12) Patent Application Publication
(10) Pub. No.: US 2006/0166290 A1
(43) Pub. Date: Jul. 27, 2006

(54) Primary central nervous system tumor specific BEHAB isoforms

(75) Inventors: Susan Hockfield, Cambrige, MA (US); Russell T. Matthews, Manchester, CT (US); Mariano S. Viapiano, New Haven, CT (US)

Correspondence Address: MORGAN, LEWIS & BOCKIUS LLP, 1701 MARKET STREET, PHILADELPHIA, PA, 19103-2921, US

(73) Assignee: Yale University

(21) Appl. No.: 11/036,255

(22) Filed: Jan. 14, 2005

Related U.S. Application Data
(60) Provisional application No. 60/536,594, filed on Jan. 15, 2004.

(51) Int. Cl. G01N 33/574; C07K 14/82; C07K 16/30

(52) U.S. Cl. 435/7.23; 530/395; 530/388.8

(57) ABSTRACT

The present invention comprises compositions and methods related to a glycosylation-variant BEHAB polypeptide, a poly-sialyated BEHAB polypeptide, and methods of use thereof.

### CROSS-REFERENCE TO RELATED APPLICATIONS

The present application is entitled to priority under 35 U.S.C. §119(e) to U.S. Provisional Application No. 60/536,594, filed on Jan. 15, 2004, which is hereby incorporated by reference in its entirety.

### STATEMENT REGARDING FEDERALLY SPONSORED RESEARCH OR DEVELOPMENT

This invention was supported in part by funds obtained from the U.S. Government (National Institutes of Health Grant Numbers RO1 NS35228 and EY06511) and the U.S. Government may therefore have certain rights in the invention.

### BACKGROUND OF THE INVENTION

Gliomas are glial tumors derived from astrocytic, oligodendroglial and ependymal cells. Gliomas are notoriously deadly brain tumors characterized by their diffuse invasion into the surrounding normal brain tissue. Further, gliomas constitute the most common form of primary CNS tumors and include several histologically distinct subtypes, most of them malignant and highly invasive (Kleihues et al., 2002, J. Neuropathol Exp Neurol, 61: 215-229). The most dangerous property of malignant gliomas is their highly invasive phenotype, which makes these primary brain tumors difficult to control and impossible to completely remove by surgery, thus accounting for the high lethality of gliomas (Pilkington, 1996, Braz J Med Biol Res, 29: 1159-72; Giese and Westphal, 1996, Neurosurgery 39: 235-252). Glioblastomas, the most common and most aggressive class of gliomas, result in patient's death typically within one year of diagnosis, due to the inevitable recurrence even after extensive resection (Bernstein and Woodard, 1995, Neurosurgery, 36: 124-132, 1995). Despite considerable advances in the understanding of these tumors, the survival rates for patients with gliomas have remained essentially unchanged for 25 years (Berens and Giese, 1999, Neoplasia, 1: 208-19). Novel therapeutic strategies will follow from an understanding of the mechanisms and molecules involved in glioma invasion.

The invasive behavior of glioma cells in the central nervous system (CNS) is quite unusual, in that the adult CNS is highly restrictive to cell movement even for non-glial tumors that metastasize to the brain (Pilkington, 1997, Anticancer Res. 17: 4103-4105; Subramanian et al., 2002, Lancet Oncol. 3: 498-507). The unique ability of gliomas to infiltrate and invade the surrounding normal neural tissue indicates that these cells are able to overcome the normal barriers to cell movement in the CNS (Giese and Westphal, 1996, Neurosurgery 39: 235-252).

One of the major barriers to cell movement in all tissues, including the CNS, is the extracellular matrix (ECM). The ECM of the CNS is composed of a hyaluronic acid (HA) scaffold associated to glycoproteins and proteoglycans (Celio and Blumcke, 1994, Brain Res Brain Res Rev. 19: 128-45). Classical fibrous ECM proteins such as laminin, type IV collagen, fibronectin and vitronectin are limited to vascular basal membranes and the glia limitans in the adult CNS and are essentially absent from the parenquima (Gladson, 1999, J. Neuropathol Exp Neurol, 58: 1029-40). Interaction of glioma cells with this HA-based ECM is mediated by several cell surface receptors such as CD44, RHAMM and proteoglycans members of the lectican family (Goldbrunner et al., 1999, Acta Neurochir (Wien) 141: 295-305; Novak and Kaye, 2000, J. Clin Neurosci, 7: 280-90; Akiyama et al., 2001, J. Neurooncol. 53: 115-27) including BEHAB/brevican (BEHAB) (Yamaguchi, 2000, Cell Mol Life Sci. 57: 276-89).

BEHAB is a CNS-specific extracellular chondroitin sulfate proteoglycan that is expressed in a spatially- and temporally-regulated manner in the mammalian brain (Jaworski et al., 1994, J. Cell Biol. 125: 495-509). BEHAB expression is upregulated in the ventricular zone coincident with gliogenesis (Jaworski et al., 1995, J. Neurosci. 15: 1352-1362), and during reactive gliosis after a stab injury (Jaworski et al., 1999, Exp Neurol. 157: 327-37), indicating that this proteoglycan is involved in glial cell proliferation and/or motility. Consistent with these findings, BEHAB mRNA expression is also dramatically upregulated in surgical samples of human glioma as well as in a rodent glioma model (Jaworski et al., 1996, Cancer Res. 56: 2293-2298). Further, BEHAB upregulation and its subsequent proteolytic processing contribute to the invasive phenotype of glioma (Zhang et al., 1998, J. Neurosci. 18: 2370-2376; Nutt et al., 2001, Neuroscientist, 7: 113-122). However, a further understanding of the molecular interactions and mechanisms through which the functions of BEHAB are mediated is still required.

One of the difficulties in characterizing the functions of BEHAB is the molecular complexity of this protein. Different isoforms of BEHAB have been described, resulting from alternative splicing (Seidenbecher et al., 1995, J. Biol. Chem. 270: 27206-27212), proteolytic cleavage (Nakamura et al., 2000, J. Biol. Chem., 275: 38885-38890; Matthews et al., 2000, J. Biol. Chem. 275: 22695-22703), and/or differential glycosylation of the core protein (Yamada et al., 1994, J. Biol. Chem. 269: 10119-10126; Viapiano et al., 2003, J. Biol. Chem., 278: 33239-33247). It is likely that these isoforms may interact differently with the cell surface and with other ECM components, and thus play unique roles in glioma progression.

A novel glycoform of BEHAB/brevican, named rat glycosylation-variant BEHAB (B/b₁₃₀), which is underglycosylated and highly expressed early in development, was discovered in the rat brain (Id.). Importantly, this isoform is the major BEHAB isoform upregulated in a rat experimental model of invasive glioma.

Almost all cancers are characterized by aberrant glycosylation of cell surface proteins (Hakomori, 2002, Proc. Natl. Acad Sci USA 99: 10231-10233). Changes in glycosylation disrupt the normal protein-protein interactions and therefore can be associated to tumor invasion and metastasis (Kim and Varki, 1997, Glycoconj. J. 14: 569-576; Gorelik et al., 2001, Cancer Metastasis Rev. 20: 245-277).

Aberrant glycosylation is identified by the appearance of either truncated versions of normal oligosaccharides or unusual types of terminal oligosaccharide sequences (e.g., Lewis x/a). These changes may equally affect N- and O-linked oligosaccharides (Burchell et al., 2001, J. Mammary Gland Biol Neoplasia 6: 355-364 2001; Dwek et al., 2001, Proteomics 1: 756-62). In particular, a general increase in the appearance of alpha 2,6- and alpha 2,3-linked sialic acid is a common feature of tumors (Narayanan, 1994, Ann Clin Lab Sci. 24: 376-384), including glioma (Reboul et al., 1996, Glycoconj. J. 13: 69-79; Yamamoto et al., 1997, Brain Res., 755: 175-179), and has been associated to an increase in the metastatic ability of certain cancers.

Lack of specific oligosaccharides in tumors, though less commonly noted, has also been described (see Dennis, 1986, Cancer Res. 46: 4594-4600; Dabelsteen et al., 1991, J. Oral Pathol Med. 20: 361-368; Ciborowski and Finn, 2002, Clin. Exp Metastasis 19: 339-45). An interesting example is represented by the cell-surface receptors with aberrantly underglycosylated neo-glycoforms in CNS tumors that cannot bind their normal ligands (e.g., CD44H in neuroblastoma (Gross et al., 2001, Med. Pediatr Oncol. 36: 139-41).

Targeting tumor cells selectively through their specific cell-surface antigens is an approach that is regaining popularity as a cancer therapy. Considerable research over the past decade has made great progress in demonstrating the utility of antibody immunotherapy in the treatment of many tumor types (see Carter, 2001, Nat. Rev Cancer, 1: 118-129), including glioma (Kurpad et al., 1995, Glia 15: 244-256; Kuan et al., 2001, Endocr. Relat Cancer, 8: 83-96; Goetz et al., 2003, J. Neurooncol. 62: 321-328). Given the refractory properties of gliomas to traditional chemo- and radiotherapy, immunotherapy is a promising treatment for primary CNS tumors. However, a hurdle in using this approach as a therapy for glioma has been the lack of good cellular targets that are both restricted to the tumor cells and available at the cell surface for targeting (Yang et al., 2003, Cancer Control. 10: 138-147). Among those that have been proposed (Kurpad et al., 1995, Glia 15: 244-256), and clinically explored are the deletion mutant EGF receptor (EGFRvIII, reviewed in Kuan et al., (2001, Endocr. Relat Cancer, 8: 83-96)), which is expressed in ˜50% of all glioblastomas (Kurpad et al., 1995, Glia 15: 244-256), and the extracellular matrix protein tenascin-C, which is highly upregulated in >90% of all gliomas compared to normal brain (McLendon et al., 2000, J. Histochem Cytochem. 48: 1103-1110).

Given the high mortality associated with primary CNS tumors, such as glioma, and the paucity of effective therapies, there exists a long felt need for molecular targets on primary CNS tumors to aid in the diagnosis and treatment of these neoplasias. The present invention meets this need.

### BRIEF SUMMARY OF THE INVENTION

The present invention encompasses a novel poly-sialyated human BEHAB isoform and methods of detecting glioma in a mammal, methods of differentially diagnosing a malignant glioma from a benign glioma, methods for assessing tumor progression and kits for detecting and diagnosing a glioma.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image depicting SEQ ID NO: 1, a peptide.

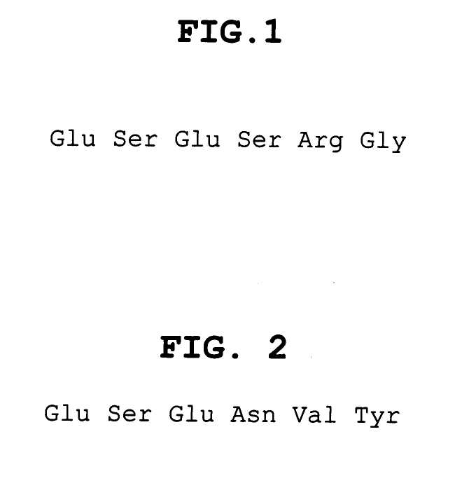

FIG. 2 is an image depicting SEQ ID NO: 2, a peptide.

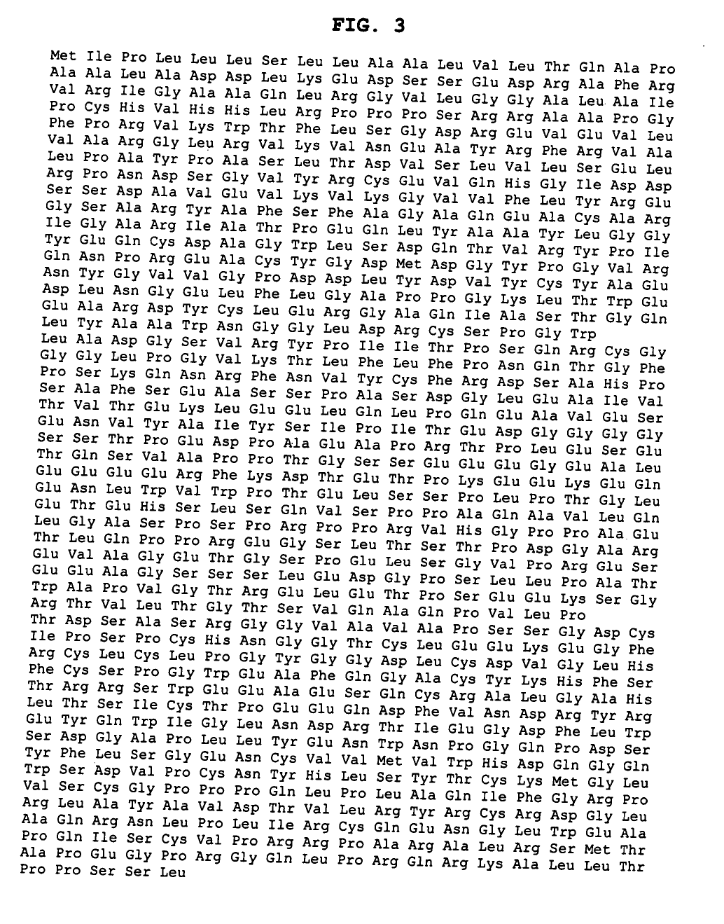

FIG. 3 is an image depicting SEQ ID NO: 3, a mammalian mutant BEHAB polypeptide.

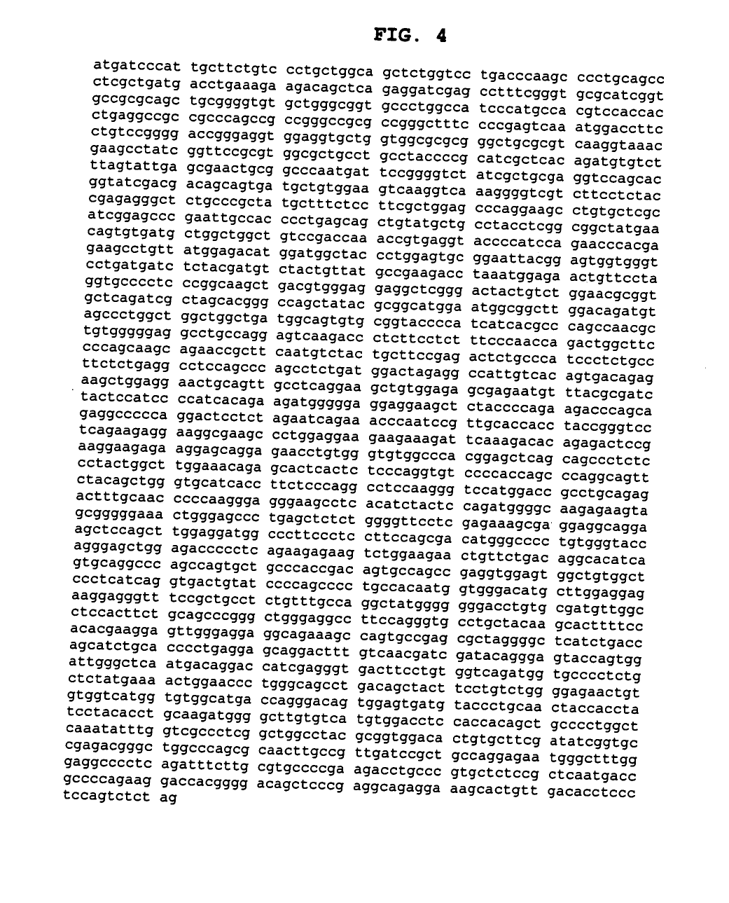

FIG. 4 is an image depicting SEQ ID NO: 4, a mammalian mutant BEHAB nucleic acid.

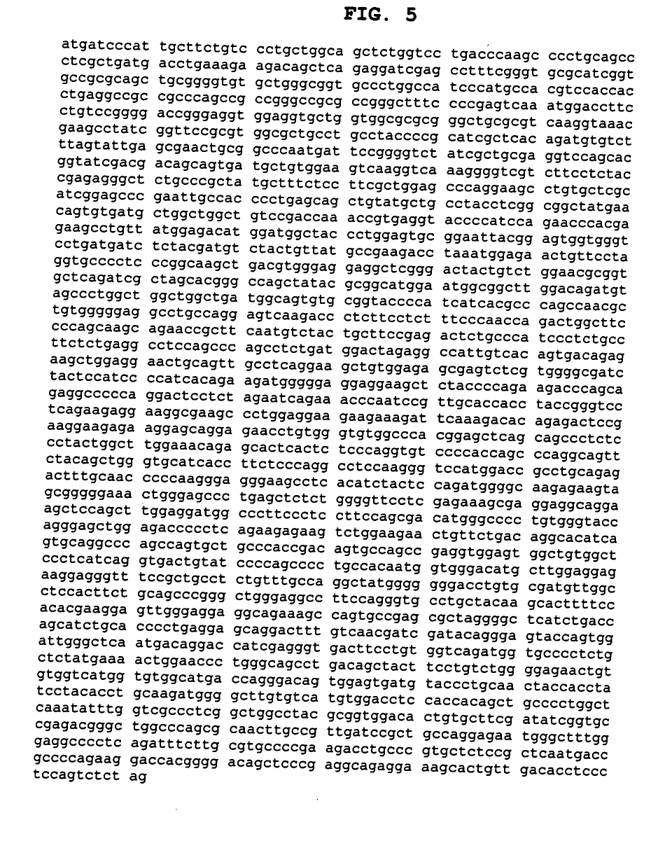

FIG. 5 is an image depicting SEQ ID NO: 5, a rat BEHAB nucleic acid.

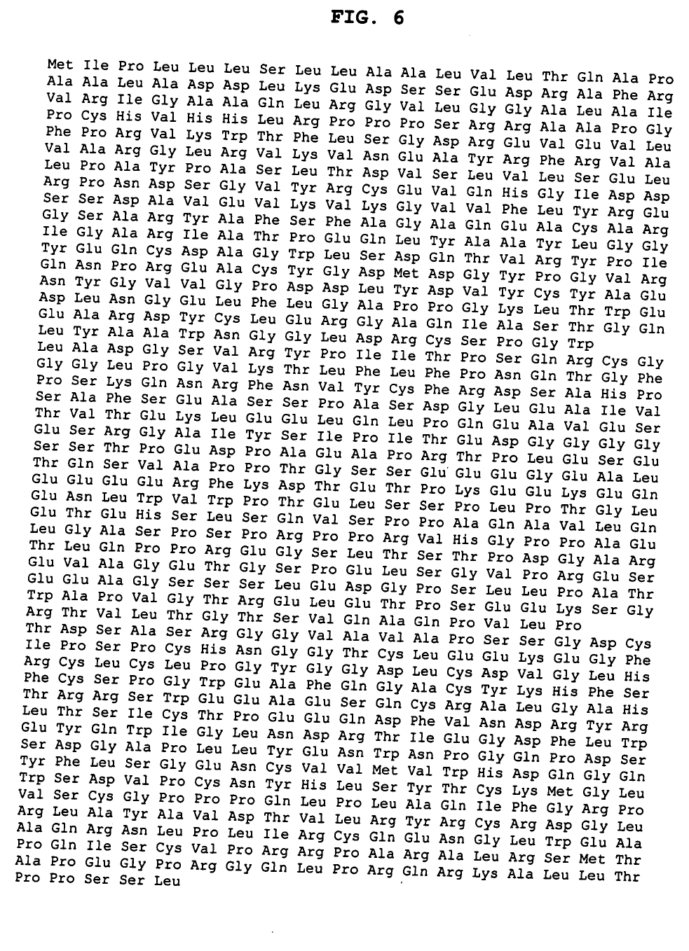

FIG. 6 is an image depicting SEQ ID NO: 6, a rat BEHAB polypeptide.

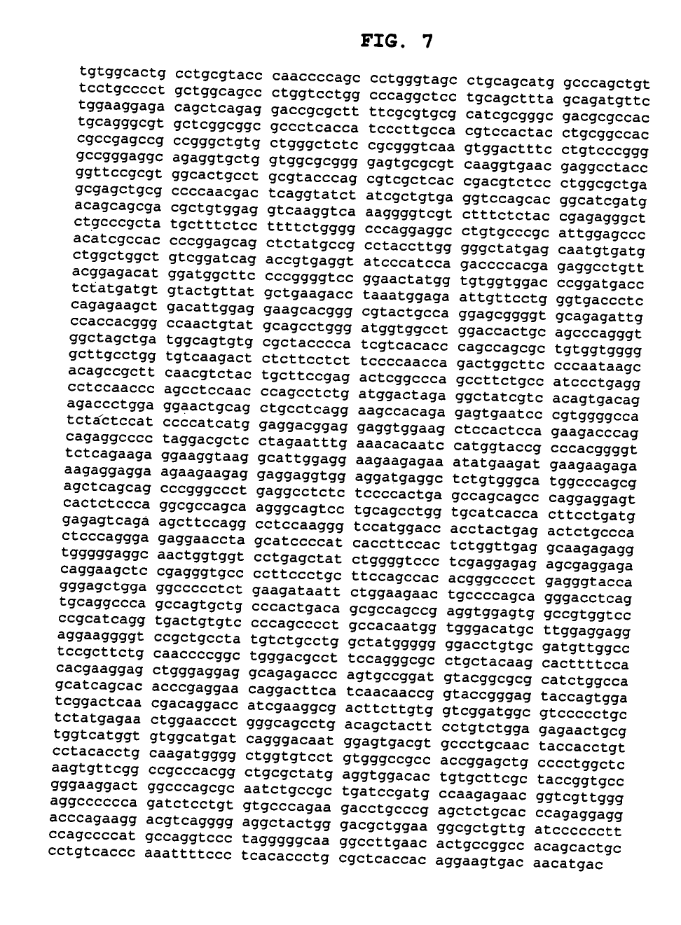

FIG. 7 is an image depicting SEQ ID NO: 7, a human BEHAB nucleic acid.

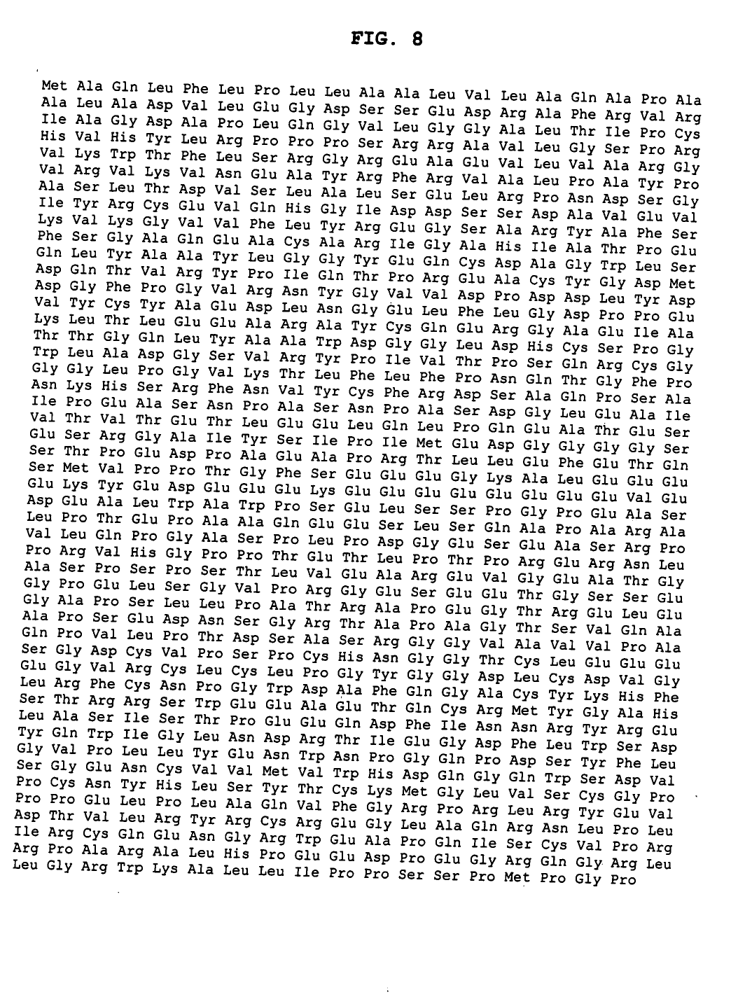

FIG. 8 is an image depicting SEQ ID NO: 8, a human BEHAB polypeptide.

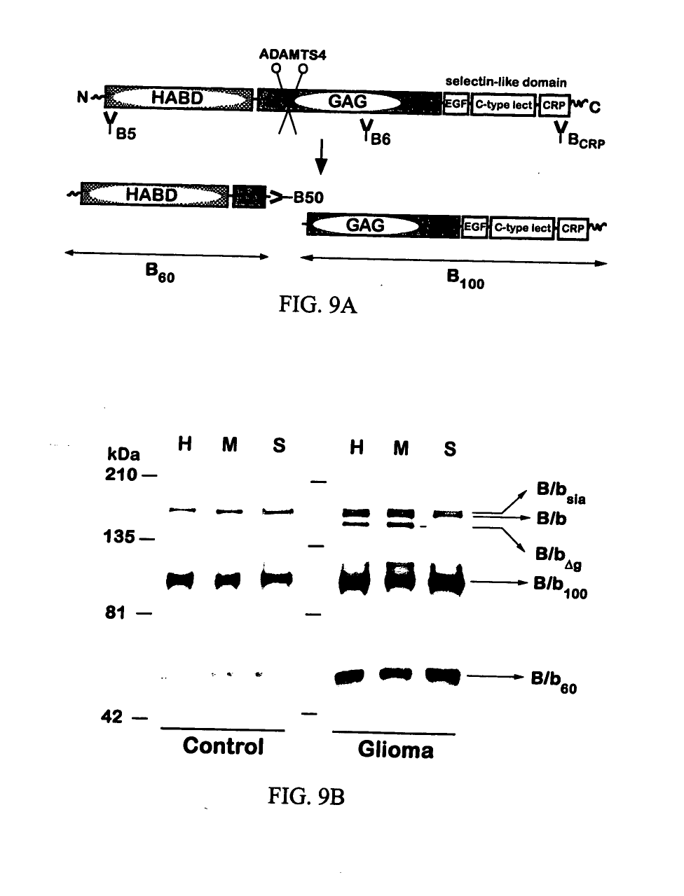

FIG. 9, comprising FIGS. 9A and 9B, is a series of images depicting a BEHAB isoform in human glioma. FIG. 9A is a schematic representation of the structure of full-length BEHAB, its cleavage products by ADAMTS-4 and the location of the epitopes recognized by the antibodies B6, B5, BCRP and B50. HABD, HA-binding domain; GAG, chondroitin sulfate attachment region; EGF, epidermal growth factor repeat; Lect, C-type lectin-like domain; CRP, complement regulatory protein-like domain. FIG. 9B is an image of a Western blot depicting the presence of glycosylation-variant BEHAB (B/b_{Δg}) and poly-sialyated BEHAB (B/bₛᵢₐ) in gliomas but not in normal brain tissue. Total homogenate (H), membrane-enriched (M) and soluble (S) fractions from a representative glioblastoma multiforme (Glioma) and a normal, age-matched brain cortex (Control) were analyzed by Western blotting. Arrows in the upper panel indicate the positions of the full-length BEHAB (B/b) and two glioma-specific isoforms: poly-sialyated BEHAB and glycosylation-variant BEHAB. Arrows in the middle and lower panels indicate the C-terminal (˜100-kDa; B/b₁₀₀) and N-terminal (˜60-kDa; B/b₆₀) cleavage products of BEHAB.

FIG. 10, comprising FIGS. 10A through 10C, is a series of images depicting poly-sialyated BEHAB and glycosylation-variant BEHAB restriction to malignant gliomas. FIG. 10A is an image depicting the expression of BEHAB isoforms in a representative subset of high-grade gliomas (grades III-IV) and age-matched controls. Asterisks indicate the position of poly-sialyated BEHAB. FIG. 10B is a graph depicting densitometry quantification of BEHAB expression from the samples depicted in FIG. 10A. Total non-cleaved BEHAB equals full-length BEHAB+poly-sialyated BEHAB+glycosylation-variant BEHAB. FIG. 10C is an image of a Western blot depicting BEHAB expression in total homogenates from other neuropathologies. Only full-length BEHAB was detected in individuals with epilepsy (epilepsy) and Alzheimer's disease (AD). BEHAB was not observed in epidermoid tumor (epid), meningioma (meng), acoustic neuroma (neur) and medulloblastoma (medul) tissue.

FIG. 11, comprising FIGS. 11A and 11B, is a series of images depicting the lack of expression of glycosylation-variant BEHAB in a subset of low-grade gliomas associated with chronic epilepsy. FIG. 1I A is an image depicting total homogenates from grade II oligodendrogliomas (n=6) analyzed by Western blotting with the B6 antibody. Samples corresponded to patients diagnosed with or without tumor-associated chronic epilepsy (c.e.). Age-matched controls are depicted to compare normal expression of full-length BEHAB at similar ages. FIG. 11B is an image depicting full-length BEHAB expression, but not glycosylation-variant BEHAB expression in surgical samples from oligodendrogliomas with benign or malignant (i.e., invasive and recurrent) clinical courses.

FIG. 12, comprising FIGS. 12A through 12C, is a series of images depicting the expression of glycosylation-variant BEHAB in early human brain development. FIGS. 12A and 12B are a series of images depicting total homogenates from human brain cortex at (1-76 years). Full-length BEHAB is the only form of BEHAB detected throughout development. FIG. 12C is an image depicting total homogenates from human cortex over prenatal and early postnatal development (16 gestational weeks to 1 year of age), and a representative surgical sample of glioblastoma (Glioma). Glycosylation-variant BEHAB is detectable in the membrane-containing fraction of normal human brain during early development, but at much lower levels than in glioma.

FIG. 13, comprising FIGS. 13A and 13B, is a series of images depicting that glycosylation-variant BEHAB is a full-length isoform of BEHAB. FIG. 13A is an image depicting solubilized brain membranes (M) from control and glioma samples immunoprecipitated in the absence (mock) or presence (B6) of B6 antibody. FIG. 13B is an image depicting culture medium (m) and cell membranes (c) from U87MG cells, transfected with full-length human BEHAB cDNA.

FIG. 14, comprising FIGS. 14A through 14C, is a series of images depicting that poly-sialyated BEHAB and glycosylation-variant BEHAB are produced by differential glycosylation of BEHAB. FIG. 14A is an image depicting soluble and particulate fractions from control and glioma samples treated with chondroitinase ABC alone (CH′ase) or with the addition of PNGase F (PNG-F), O-glycosidase (O-glycos) and sialidase. FIG. 14B is an image depicting membranes from a glioma sample and from BEHAB-transfected U87MG cells denatured (Denat) and treated with chondroitinase and PNGase-F (PNG-F). FIG. 14C is an image depicting the soluble fraction from a glioma sample expressing poly-sialyated BEHAB that was chondroitinased (ctrl) and additionally deglycosylated in native conditions with PNGase-F (PNG-F), sialidase (sialid) or sialidase plus O-glycosidase (sia/O-gly).

FIG. 15, comprising FIGS. 15A through 15K, is a series of images depicting that glycosylation-variant BEHAB is located on the cell surface. U87MG cells were transfected with the cDNA of full-length human BEHAB, either untagged (FIGS. 15A and 15B) or tagged with the V5 epitope (FIGS. 15C and 15D). Live cells were stained using the antibodies B6 (FIGS. 15A and 15B) and anti-V5 (FIGS. 15C and 15D). Negative controls included B6-staining cells transfected with control vector (FIG. 15E) and staining BEHAB-transfected cells with non-immune rabbit serum (FIG. 15F). FIGS. 15G through 15J are a series of images depicting U87MG cells transfected with V5-tagged BEHAB and live-stained with B6 antibody and V5 antibody. Cell nuclei were visualized with 4′,6-diamino-2-phenylindole (DAPI). Bar=25 μm. FIG. 15K is an image depicting BEHAB-transfected U87MG cells (B/b) expressing full-length BEHAB in the culture medium (m) and glycosylation-variant BEHAB in the cell membranes (c). Only one isoform is detected in the homogenates (homog) of these transfected cells, glycosylation-variant BEHAB. Ctrl, homogenate from control-transfected cells.

FIG. 16, is an image depicting that glycosylation-variant BEHAB associates peripherally with cell membranes in a calcium-independent manner. Total membranes (M) from control and glioma samples were resuspended with (EDTA) or without (Tris). Parallel samples were resuspended in Na₂CO3 (Na₂CO3). S equals supernatant, P equals pellet.

### DETAILED DESCRIPTION OF THE INVENTION

The present invention is based on the discovery of a novel underglycosylated or unglycosylated BEHAB isoform in human brain termed glycosylation-variant BEHAB, which, as demonstrated by the data disclosed herein, is absent from the normal adult brain and neuropathologic controls and is highly over-expressed in surgical samples from human glioblastoma. Further, glycosylation-variant BEHAB is specific for malignant tumors and is not expressed on low-grade and benign gliomas, such as a subgroup of oligodendriogliomas. Thus, the present invention provides methods for the differential diagnosis of various gliomas.

As evidenced by the data disclosed elsewhere herein, human glycosylation-variant BEHAB is an under- or unglycosylated isoform that lacks most, if not all, of the carbohydrates with which it is typically invested. Despite the lack of glycosylation, human glycosylation-variant BEHAB is found on the extracellular surface of cells and binds via a mechanism unique from other known BEHAB isoforms. As disclosed elsewhere herein, glycosylation-variant BEHAB can play a unique role in glioma progression and can be a relevant cell-surface target for immuno-therapy.

The present invention further encompasses a novel poly-sialyated BEHAB molecule that is present in some high-grade glioma samples, but not in other neuropathologies and specific low grade gliomas. Thus, the present invention includes compositions comprising a poly-sialyated BEHAB molecule and methods of detecting and differentiating high-grade gliomas using poly-sialyated BEHAB.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Amplification” refers to any means by which a polynucleotide sequence is copied and thus expanded into a larger number of polynucleotide molecules, e.g., by reverse transcription, polymerase chain reaction, and ligase chain reaction.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator” as the term is used herein, is meant any device including, but not limited to, a hypodermic syringe, a pipette, and the like, for administering the mutant BEHAB nucleic acid, protein, and/or anti-BEHAB antibodies and the antisense BEHAB nucleic acid of the invention to a mammal.

“BEHAB” “full-length BEHAB”, or “endogenous BEHAB” as the terms are used synonymously herein, refers to the Brain-Enriched Hyaluronan Binding molecule, otherwise known as brevican. Full-length BEHAB has a molecular weight of greater than about 150 kDa in rats and mice, and greater than about 160 kDa, but less than 163 kDa in humans and is exemplified by the nucleotide and amino acid sequences set forth in SEQ ID NO: 5 and SEQ ID NO: 6 for rat full-length BEHAB, and SEQ ID NO: 7 and SEQ ID NO: 8 for human full-length BEHAB, respectively.

“Biological sample,” as that term is used herein, means a sample obtained from or in a mammal that can be used to assess the level of expression of a BEHAB, the level of BEHAB protein present, or both. Such a sample includes, but is not limited to, a blood sample, a neural tissue sample, a brain sample, and a cerebrospinal fluid sample.

“Cleavage” is used herein to refer to the disassociation of a peptide bond between two amino acids in a polypeptide, thereby separating the polypeptide comprising the two amino acids into at least two fragments.

A “cleavage inhibitor” is used herein to refer to a molecule, compound or composition that prevents the cleavage of a polypeptide either by titrating the protease responsible for cleavage, blocking the cleavage site, or otherwise making the cleavage site unrecognizable to a protease.

“Cleavage inhibiting amount” is used herein to refer to an effective amount of a cleavage inhibitor.

“Cleavage products” is used herein to refer to the fragments of an initial polypeptide resulting from the cleavage of the initial polypeptide into two or more fragments. As an example, the cleavage products of the 145 kDa BEHAB protein include 90 kDa and 50 kDa fragments.

By “complementary to a portion or all of the nucleic acid encoding BEHAB” is meant a sequence of nucleic acid which does not encode a BEHAB protein. Rather, the sequence which is being expressed in the cells is identical to the non-coding strand of the nucleic acid encoding a BEHAB protein and thus, does not encode BEHAB protein.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g. amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A first region of an oligonucleotide “flanks” a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 500 nucleotides, even more preferably, at least about 500 nucleotides to about 1000 nucleotides, yet even more preferably, at least about 1000 to about 1500, even more preferably, at least about 1500 nucleotides to about 2000 nucleotides, yet even more preferably, at least about 2000 to about 2500, even more preferably, at least about 2500 nucleotides to about 2600 nucleotides, yet even more preferably, at least about 2600 to about 2650, and most preferably, the nucleic acid fragment will be greater than about 2652 nucleotides in length.

As applied to a protein, a “fragment” of BEHAB is about 20 amino acids in length. More preferably, the fragment of a BEHAB is about 100 amino acids, even more preferably, at least about 200, yet more preferably, at least about 300, even more preferably, at least about 400, yet more preferably, at least about 500, even more preferably, about 600, and more preferably, even more preferably, at least about 700, yet more preferably, at least about 800, even more preferably, about 850, and more preferably, at least about 884 amino acids in length.

As used herein, a “glycosylation-variant BEHAB isoform” and “glycosylation-variant BEHAB” means a BEHAB protein having an altered glycosylation pattern as compared to the glycosylation pattern of full-length BEHAB and a molecular weight less than about 150 kDa in rats and less than about 160 kDa in humans. The term glycosylation-variant BEHAB isoform or glycosylation-variant BEHAB includes underglycosylated BEHAB, differently-glycosylated BEHAB and unglycosylated BEHAB.

As used herein, a “poly-sialyated full-length BEHAB” and “poly-sialyated full-length BEHAB isoform” means a BEHAB protein having an altered glycosylation pattern as compared to the glycosylation pattern of full-length BEHAB and a molecular weight greater than about 160 kDa in humans when not treated with sialidase.

As used herein, a “differently-glycosylated BEHAB” and a “differently-glycosylated BEHAB isoform” refers to a BEHAB protein having an altered glycosylation pattern wherein the carbohydrate and sugar content is similar to that of full-length BEHAB, but the composition of the sugars associated with the amino acid backbone is altered.

“Underglycosylated BEHAB isoform” and “underglycosylated BEHAB” are used herein to refer to a BEHAB protein having the primary amino acid sequence of a full-length BEHAB protein, or a fragment thereof, but having less than the glycosylation content of the full-length BEHAB protein, but still having at least one sugar or carbohydrate associated with the protein.

“Unglycosylated BEHAB isoform” and “unglycosylated BEHAB” are used herein to refer to a BEHAB protein having the primary amino acid sequence of a full-length BEHAB protein, or fragment thereof, but having no sugars or carbohydrates associated with the protein.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

A “malignant high grade glioma” is used herein to refer to a grade III or grade IV glioma using the histologic grading system used to grade gliomas and the level of differentiation in glioma cells and tissues. A “benign low grade glioma” is used herein to refer to a grade I or grade II glioma using the histologic grading system used to grade gliomas and the level of differentiation in glioma cells and tissues. Grade II gliomas are well-differentiated (low grade), Grade II gliomas are moderately differentiated (intermediate grade), Grade III gliomas are poorly differentiated (high grade) and Grade IV gliomas are undifferentiated (high grade). The criteria for grading gliomas are those according to the American Joint Committee on Cancer. AJCC Cancer Staging Manual. 6th ed. New York, N.Y.: Springer, 2002.

“Mutant BEHAB” is used herein to refer to a Brain Enriched Hyaluronan Binding molecule in which the amino acid sequence has been modified to inhibit cleavage by proteases.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is naturally-occurring.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

“Primary CNS tumor” is used herein to refer to a neoplasia with origins in the brain, in that the cancerous cells did not originate in another part of the body and metastasize to the brain. Examples of primary CNS tumors include, but are not limited to, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, reactive gliosis, primitive neuroectodermal tumors, schwannomas, lymphomas, vascular tumors, and lymphomas.

“Treating a primary CNS tumor” is used herein to refer to a situation where the severity of a symptom of a primary CNS tumor, including the volume of the tumor or the frequency with which any symptom or sign of the tumor is experienced by a patient, or both, is reduced, or where time to tumor progression or survival time is increased.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By the term “specifically binds,” as used herein, is meant an antibody which recognizes and binds an epitope of a BEHAB protein, but does not substantially recognize or bind other molecules in a sample.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

A “transgene”, as used herein, means an exogenous nucleic acid sequence comprising a nucleic acid which encodes a promoter/regulatory sequence operably linked to nucleic acid which encodes an amino acid sequence, which exogenous nucleic acid is encoded by an animal or cell.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Description

I. Isolated Polypeptides

The invention includes an isolated polypeptide comprising a poly-sialyated BEHAB molecule. Preferably, the polypeptide is about 1% homologous, more preferably, about 5% homologous, even more preferably about 10% homologous, even more preferably about 20% homologous, even more preferably, the nucleic acid is about 30% homologous, more preferably, about 40% homologous, even more preferably about 50% homologous, even more preferably, the nucleic acid is about 60% homologous, more preferably, about 70% homologous, even more preferably about 80% homologous. Preferably, the nucleic acid is about 90% homologous, more preferably, about 95% homologous, even more preferably about 99% homologous, even more preferably about 99.9% homologous to SEQ ID NO: 8, disclosed herein. Even more preferably, the polypeptide is SEQ ID NO: 8.

The poly-sialyated BEHAB polypeptide of the present invention is from about 2 to about 7 kilodaltons larger than full-length BEHAB, more preferably from about 3 to about 6 kilodaltons larger than full-length BEHAB, more preferably about 4 to about 5 larger than full-length BEHAB. Most preferably, the total weight of a poly-sialyated BEHAB polypeptide is from about 163 to about 166 kilodaltons.

The poly-sialyated BEHAB polypeptide of the present invention comprises about 9 to about 21 additional sialic acid residues compared to full-length BEHAB, more preferably from about 9 to about 21 additional sialic acid residues compared to full-length BEHAB, about 10 to about 20 additional sialic acid residues compared to full-length BEHAB, about 11 to about 19 additional sialic acid residues compared to full-length BEHAB, about 12 to about 18 additional sialic acid residues compared to full-length BEHAB, about 13 to about 17 additional sialic acid residues compared to full-length BEHAB, about 14 to about 16 additional sialic acid residues compared to full-length BEHAB, about 15 additional sialic acid residues compared to full-length BEHAB.

As demonstrated by the data disclosed herein, the poly-sialyated BEHAB polypeptide of the present invention comprises additional sialic acid residues attached via an O-linkage and not an N-linkage.

The present invention also provides for analogs of proteins or peptides which comprise a poly-sialyated BEHAB molecule as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “derivatives,” and “variants” of the peptides of the invention (or of the DNA encoding the same) which derivatives and variants are poly-sialyated BEHAB peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the poly-sialyated BEHAB peptide of the present invention.

The biological/biochemical properties of a poly-sialyated BEHAB molecule are disclosed elsewhere herein.

The skilled artisan would understand, based upon the disclosure provided herein, that poly-sialyated BEHAB biological activity encompasses, but is not limited to, the ability of a molecule to be expressed in glioma, to be detected in glioma, to be expressed on a cell surface, and the like.

III. Vectors

In other related aspects, the invention includes an isolated nucleic acid encoding a poly-sialyated BEHAB operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Expression of poly-sialyated BEHAB, either alone or fused to a detectable tag polypeptide, in cells which either normally express full-length BEHAB or do not express BEHAB, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of the nucleic acid encoding poly-sialyated BEHAB may be accomplished by placing the nucleic acid encoding poly-sialyated BEHAB, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Expressing poly-sialyated BEHAB using a vector allows the isolation of large amounts of recombinantly produced protein.

The invention includes not only methods of producing poly-sialyated BEHAB, but it also includes methods relating to detecting glycosylation-variant BEHAB expression, including poly-sialyated BEHAB expression, protein level, and/or activity since detecting glycosylation-variant BEHAB expression, and/or activity or decreasing glycosylation-variant BEHAB expression and/or activity can be useful in providing effective therapeutics.

Selection of any particular plasmid vector or other DNA vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention thus includes a vector comprising an isolated nucleic acid encoding a human poly-sialyated BEHAB. The incorporation of a desired nucleic acid into a vector and the choice of vectors is well-known in the art as described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention also includes cells, viruses, proviruses, and the like, containing such vectors. Methods for producing cells comprising vectors and/or exogenous nucleic acids are well-known in the art. See, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

A nucleic acid encoding poly-sialyated BEHAB may be cloned into various plasmid vectors. However, the present invention should not be construed to be limited to plasmids or to any particular vector. Instead, the present invention should be construed to encompass a wide plethora of vectors which are readily available and/or well-known in the art.

IV. Recombinant Cells

The invention further includes a method of making a poly-sialyated BEHAB isoform in a recombinant cell comprising, inter alia, an isolated nucleic acid encoding a poly-sialyated BEHAB protein. That is, a poly-sialyated BEHAB isoform can be produced in a recombinant cell by transfecting a cell with an isolated nucleic acid encoding poly-sialyated BEHAB, or a fragment thereof, and isolating the poly-sialyated BEHAB isoform therefrom. Further, methods for transfecting a cell and producing a protein therefrom are well known in the art and are described in detail elsewhere herein. Recombinant cells thus include those which express full-length BEHAB, and those that express a glycosylation-variant BEHAB, such as poly-sialyated BEHAB.

The invention should be construed to include any cell type into which a nucleic acid encoding a poly-sialyated BEHAB is introduced, including, without limitation, a prokaryotic cell and a eukaryotic cell comprising an isolated nucleic acid encoding poly-sialyated BEHAB.

The invention includes a eukaryotic cell which, when the recombinant gene of the invention is introduced therein, and the protein encoded by the desired gene is expressed therefrom, where it was not previously present or expressed in the cell or where it is now expressed at a level or under circumstances different than that before the transgene was introduced, a benefit is obtained. Such a benefit may include the fact that there has been provided a system wherein the expression of the desired gene can be studied in vitro in the laboratory or in a mammal in which the cell resides, a system wherein cells comprising the introduced gene can be used as research, diagnostic and therapeutic tools, and a system wherein mammal models are generated which are useful for the development of new diagnostic and therapeutic tools for selected disease states in a mammal.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid encoding full-length BEHAB and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all of, for example, poly-sialyated BEHAB (i.e., a “knock-out” vector) or which insert (i.e., a “knock-in” vector) a nucleic acid encoding poly-sialyated BEHAB, or a fragment thereof, from or into a mammalian genome, respectively. The crucial feature of the targeting vector is that it comprise sufficient portions of two sequences located towards opposite, i.e., 5′ and 3′, ends of the BEHAB open reading frame (ORF) in the case of a “knock-out” vector, to allow deletion/insertion by homologous recombination to occur such that all or a portion of the nucleic acid encoding BEHAB is deleted from a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the BEHAB coding region to be used in the targeting vector may be easily selected based upon known methods and following the teachings disclosed herein based on the disclosure provided herein including the nucleic and amino acid sequences of poly-sialyated BEHAB. Armed with these sequences, one of ordinary skill in the art would be able to construct the transgenes and knock-out vectors of the invention.

Methods and compositions useful for maintaining mammalian cells in culture are well known in the art, wherein the mammalian cells are obtained from a mammal including, but not limited to, cells obtained from a mouse, a rat, a human, and the like.

The recombinant cell of the invention can be used to study the effect of qualitative and quantitative alterations in poly-sialyated BEHAB and/or glycosylation-variant BEHAB levels on tumor progression and invasiveness. This is because the fact that BEHAB is secreted and possesses a hyaluronan binding domain indicates that BEHAB is involved in the function, composition, or activity of the ECM. Further, the recombinant cell can be used to produce poly-sialyated BEHAB and/or glycosylation-variant BEHAB for use for therapeutic and/or diagnostic purposes. That is, a recombinant cell expressing poly-sialyated BEHAB and/or glycosylation-variant BEHAB can be used to produce large amounts of purified and isolated poly-sialyated BEHAB and/or glycosylation-variant BEHAB that can be used in the diagnosis of gliomas and the differential diagnosis of gliomas, including, but not limited to, distinguishing between benign and malignant tumors, distinguishing between subgroups of benign and malignant oligodendriogliomas and astrocytomas, and the like.

One skilled in the art would appreciate, based upon this disclosure, that cells comprising decreased levels of poly-sialyated BEHAB protein, decreased levels of BEHAB and/or BEHAB cleavage product activity, or both, include, but are not limited to, cells expressing inhibitors of BEHAB expression (e.g., antisense or ribozyme molecules, synthetic antibodies or intrabodies).

Further the present invention comprises inhibition of a gene expressing BEHAB, a glycosylation-variant BEHAB, a poly-sialyated BEHAB, or fragments thereof. The present invention can be achieved through the use of interfering RNA. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., Nature (1998) 391(19):306-311; Timmons et al., Nature (1998) 395:854; Montgomery et al., TIG (1998) 14(7):255-258; David R. Engelke, Ed., RNA Interference (RNAi) Nuts & Bolts of RNAi Technology, DNA Press (2003); and Gregory J. Hannon, Ed., RNAi A Guide to Gene Silencing, Cold Spring Harbor Laboratory Press (2003). Therefore, the present invention also includes methods of silencing the gene encoding BEHAB, a glycosylation-variant BEHAB, a poly-sialyated BEHAB, or fragments thereof by using RNAi technology.

V. Antibodies

Also included is an antibody that specifically binds BEHAB, a glycosylation-variant BEHAB, a poly-sialyated BEHAB, or fragments thereof.

The skilled artisan, when equipped with the present disclosure, would also understand that the present invention further comprises antibodies that bind a glycosylation-variant BEHAB isoform, including an underglycosylated BEHAB isoform and an unglycosylated BEHAB isoform and a poly-sialyated BEHAB isoform. The generation of antibodies is described elsewhere herein, and their production is accomplished using techniques and skills well known in the art. Antibodies that bind glycosylation-variant BEHAB, including underglycosylated BEHAB, unglycosylated BEHAB and poly-sialyated BEHAB include, but are not limited to the B5, B6 and BCRP antibodies described in the experimental details herein and elsewhere in the art (Matthews et al., 2000, J. Biol. Chem. 275: 22695-22703). Further, the antibodies described herein can bind various forms of mammalian BEHAB, including rat and human, and art thus useful in the present invention for the detection, diagnosis, and treatment of primary CNS tumors associated with BEHAB.

The present invention is not limited to the antibodies enumerated herein, but rather also includes anti-glycosylation-variant BEHAB antibodies discovered and generated in the future as well as anti-poly-sialyated BEHAB antibodies discovered and/or generated in the future. An antibody to a glycosylation-variant BEHAB, including a differently-glycosylated, underglycosylated and unglycosylated BEHAB, as well as a poly-sialyated BEHAB, can be generated in a variety of ways well known in the art. As a non-limiting example, a nucleic acid encoding BEHAB, or a fragment thereof, can be transformed into an organism that does not glycosylate the proteins it produces, such as E. coli. Methods for the production of proteins in E. coli and other prokaryotic species are well known in the art and are described elsewhere herein. The protein isolated from a non-glycosylating prokaryotic species can then be administered to a mammal to generate antibodies, as is described herein. The antibodies specifically bind a glycosylation-variant BEHAB isoform, including unglycosylated BEHAB.

Further, antibodies to glycosylation-variant BEHAB, including poly-sialyated BEHAB can be generated by contacting a full-length BEHAB protein with glycosidases in order to remove some or all of the sugars and carbohydrates associated with the BEHAB protein backbone. In addition, full-length BEHAB can be contacted with glycosyltransferases, including glycosyltransferases known in the art to attach sialic acid to a protein. Such glycosidases and glycosyltransferases are well known in the art, and a number of relevant glycosidases are described elsewhere herein. Glycosyltransferases known in the art are described in, for example, Essentials of Glycobiology (1999, eds. Ajit Varki, et al. Cold Spring Harbor, N.Y.: Cold Spring Harbor Laboratory Press), incorporated by reference in its entirety herein. Further, the skilled artisan, when equipped with the present disclosure and the data disclosed herein, would readily be able to select specific glycosidases for the removal of a certain family of sugars or carbohydrates while optionally retaining others on sugars and carbohydrates on the BEHAB molecule. The BEHAB molecule, after treatment with a glycosidase, can then be administered to an animal for the generation of antibodies to glycosylation-variant-BEHAB. Methods for the administration of a protein to a mammal and the generation of an antibody are well known in the art and are described herein. Similarly, for the generation of antibodies specific to poly-sialyated BEHAB, glycosyltransferases, such as those that add sialic acid to protein, can be used to add sialic acid to full-length BEHAB. This molecule can then be administered to an animal using the techniques well known in the art and described elsewhere herein to generate antibodies, including monoclonal and polyclonal antibodies, to poly-sialyated BEHAB.

Alternatively, poly-sialyated and glycosylation variant BEHAB can be isolated from cells in which the molecule naturally occurs, including glioma cells, in order to produce antibodies. Methods for isolating poly-sialyated BEHAB and glycosylation-variant BEHAB from a cell or tissue are described elsewhere herein.

The invention further comprises generating antibodies specific to glycosylation-variant BEHAB. Such antibodies are useful in the compositions, methods and kits disclosed elsewhere herein. As a non-limiting example, an antibody specific to glycosylation-variant BEHAB can be generated by administering a peptide or protein comprising fragments of the primary amino acid sequence of BEHAB. Such fragments can comprise consensus glycosylation sites present in the primary amino acid sequence of BEHAB. The skilled artisan will readily recognize such consensus glycosylation sites by their sequences and amino acid content. As an example, O-linked saccharides are usually attached via a glycosidic bond on a threonine or serine residue, and in some cases, on hydroxylysine or hydroxyproline. Further, N-linked saccharides are often attached to an asparagine residue, often at a site having a sequence of any amino acid bound to an asparagine bound to any amino acid bound to threonine. Thus, the skilled routineer, when armed with the present disclosure and the methods disclosed herein, would readily be able to identify consensus glycosylation sites in a BEHAB primary amino acid sequence, generate peptides for immunizing an animal comprising these consensus glycosylation sites, and generate antibodies that specifically bind glycosylation-variant BEHAB. Such antibodies are useful in therapeutic treatments, including, but not limited to immunizing a mammal against the formation of primary CNS tumors, treating a primary CNS tumor, detecting a primary CNS tumor in a mammal either in vivo or in vitro, and other methods and uses disclosed elsewhere herein.

The antibodies of the present invention are especially useful for the diagnosis and differential diagnosis of gliomas in a mammal, including a human. This is because, as demonstrated by the data disclosed herein, antibodies against glycosylation-variant BEHAB and poly-sialyated BEHAB can be used in, for example assays to differentiate between oligodendrogliomas and other gliomas. The antibodies of the present invention can further be used to differentiate between benign tumors and malignant tumors in the CNS. Thus, the antibodies of the present invention can be used for the diagnosis of CNS tumors, such as gliomas, in a mammal, including a human.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the BEHAB portion is rendered immunogenic (e.g., BEHAB conjugated with keyhole limpet hemocyanin, KLH) and a portion comprising the respective rodent and/or human BEHAB amino acid residues. The chimeric proteins are produced by cloning the appropriate nucleic acids encoding BEHAB (e.g., SEQ ID NO: 7) into a plasmid vector suitable for this purpose, such as but not limited to, pMAL-2 or pCMX. Other methods of producing antibodies that specifically bind BEHAB and portions thereof are detailed in Matthews et al. (2000, J. Biol. Chem. 275: 22695-22703).

However, the invention should not be construed as being limited solely to polyclonal antibodies that bind a full-length BEHAB. Rather, the invention should be construed to include other antibodies, as that term is defined elsewhere herein, to mammalian BEHAB, or portions thereof. Further, the present invention should be construed to encompass antibodies that, among other things, bind to BEHAB and are able to bind BEHAB present on Western blots, in immunohistochemical staining of tissues thereby localizing BEHAB, including poly-sialyated BEHAB and/or glycosylation-variant BEHAB, in the tissues, and in immunofluorescence microscopy of a cell transiently or stably transfected with a nucleic acid encoding at least a portion of BEHAB.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian BEHAB, including poly-sialyated BEHAB and/or glycosylation-variant BEHAB. That is, the invention includes immunizing an animal using an immunogenic portion, or antigenic determinant, of the BEHAB protein, for example, the epitope comprising a glycosylation site.

The antibodies can be produced by immunizing an animal such as, but not limited to, a rabbit or a mouse, with a BEHAB protein, or a portion thereof, or by immunizing an animal using a protein comprising at least a portion of BEHAB, or a fusion protein including a tag polypeptide portion comprising, for example, a maltose binding protein tag polypeptide portion, covalently linked with a portion comprising the appropriate BEHAB amino acid residues. One skilled in the art would appreciate, based upon the disclosure provided herein, that smaller fragments of these proteins can also be used to produce antibodies that specifically bind BEHAB.

One skilled in the art would appreciate, based upon the disclosure provided herein, that various portions of an isolated BEHAB polypeptide can be used to generate antibodies to either epitopes comprising the cleavage site of BEHAB or to epitopes present on the cleavage products of BEHAB. Once armed with the sequence of BEHAB and the detailed analysis localizing the various epitopes and cleavage products of the protein, the skilled artisan would understand, based upon the disclosure provided herein, how to obtain antibodies specific for the various portions of a mammalian BEHAB polypeptide using methods well-known in the art or to be developed.

Therefore, the skilled artisan would appreciate, based upon the disclosure provided herein, that the present invention encompasses antibodies that neutralize and/or inhibit BEHAB activity, as well as antibodies that detect poly-sialyated BEHAB and/or glycosylation-variant BEHAB in a biological sample.

The invention should not be construed as being limited solely to the antibodies disclosed herein or to any particular immunogenic portion of the proteins of the invention. Rather, the invention should be construed to include other antibodies, as that term is defined elsewhere herein, to BEHAB, or portions thereof, or to proteins sharing at least some homology with a polypeptide having the amino acid sequence of SEQ ID NO: 8. Preferably, the polypeptide is about 1% homologous, more preferably, about 5% homologous, more preferably, about 10% homologous, even more preferably, about 20% homologous, more preferably, about 30% homologous, preferably, about 40% homologous, more preferably, about 50% homologous, even more preferably, about 60% homologous, more preferably, about 70% homologous, even more preferably, about 80% homologous, preferably, about 90% homologous, more preferably, about 95% homologous, even more preferably, about 99% homologous, and most preferably, about 99.9% homologous to human BEHAB (SEQ ID NO: 8).

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art and described elsewhere herein.

The invention encompasses polyclonal, monoclonal, synthetic antibodies, and the like. One skilled in the art would understand, based upon the disclosure provided herein, that the crucial feature of the antibody of the invention is that the antibody bind specifically with BEHAB, including poly-sialyated BEHAB and/or glycosylation-variant BEHAB. That is, the antibody of the invention recognizes BEHAB, or a fragment thereof (e.g., an immunogenic portion, glycosylation-variant or antigenic determinant thereof), on Western blots, in immunostaining of cells, and immunoprecipitates BEHAB, including poly-sialyated BEHAB and/or glycosylation-variant BEHAB, using standard methods well-known in the art.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (1992, Critical Rev. Immunol. 12:125-168), and in the references cited therein, and in Gu et al. (1997, Thrombosis and Hematocyst 77:755-759). The present invention also includes the use of humanized antibodies specifically reactive with epitopes of BEHAB, including poly-sialyated BEHAB and/or glycosylation-variant BEHAB. Such antibodies are capable of specifically binding BEHAB, or a fragment thereof. The humanized antibodies of the invention have a human framework and have one or more complementarity determining regions (CDRs) from an antibody, typically, but not limited to a mouse antibody, specifically reactive with BEHAB, or a fragment thereof. Thus, for example, humanized antibodies to BEHAB are useful in the detection and/or differential diagnosis of primary CNS tumors such as gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, pineal tumors, pituitary tumors, pituitary adenomas, primitive neuroectodermal tumors, vascular tumors, and the like.

When the antibody used in the invention is humanized, the antibody may be generated as described in Queen, et al. (U.S. Pat. No. 6,180,370), Wright et al., (1992, Critical Rev. Immunol. 12:125-168) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as BEHAB, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671, which is herein incorporated by reference. CDRs useful in producing the antibodies of the present invention may be similarly derived from DNA encoding monoclonal antibodies capable of binding to BEHAB, including poly-sialyated BEHAB and/or glycosylation-variant BEHAB. Such humanized antibodies may be generated using well known methods in any convenient mammalian source capable of producing antibodies, including, but not limited to, mice, rats, rabbits, or other vertebrates. Suitable cells for constant region and framework DNA sequences and host cells in which the antibodies are expressed and secreted, can be obtained from a number of sources, for example, American Type Culture Collection, Manassas, Va.

In addition to the humanized antibodies discussed above, other modifications to native antibody sequences can be readily designed and manufactured utilizing various recombinant DNA techniques well known to those skilled in the art. Moreover, a variety of different human framework regions may be used singly or in combination as a basis for humanizing antibodies directed to BEHAB, including glycosylation-variant BEHAB and poly-sialyated BEHAB. In general, modifications of genes may be readily accomplished using a variety of well-known techniques, such as site-directed mutagenesis (Gillman and Smith, Gene, 8:81-97 (1979); Roberts et al., 1987, Nature, 328:731-734).

Alternatively, a phage antibody library may be generated. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor Laboratory, New York).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (992, Critical Rev. Immunol. 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

VI. Compositions

The present invention encompasses a glycosylation-variant BEHAB isoform, including, but not limited to differently-glycosylated, underglycosylated BEHAB, poly-sialyated glycosylation variant BEHAB and unglycosylated BEHAB. The glycosylation-variant BEHAB of the present invention comprises a BEHAB molecule with altered or less than the full complement of sugars and carbohydrates found on full-length BEHAB. As disclosed by the data herein, glycosylation-variant BEHAB is the major upregulated form of BEHAB in primary CNS tumors, including, but not limited to, gliomas. Thus the present invention includes a glycosylation-variant BEHAB that is useful for, inter alia, a diagnostic tool for primary CNS tumors, a research tool for elucidating the interaction of the neural extracellular matrix with cancer-causing mutations, dysfunctions, and the like. Further, the glycosylation-variant BEHAB of the present invention is useful as a reagent in compositions, methods and kits for the detection, treatment, and diagnosis of primary CNS tumors, including, but not limited to immunotherapy of primary CNS tumors, such as glioma.

Thus the present invention includes a poly-sialyated BEHAB that is useful for, among other things, a diagnostic tool for primary CNS tumors, including gliomas, a research tool for elucidating the interaction of the neural extracellular matrix with cancer-causing mutations, dysfunctions, and the like. Further, the poly-sialyated BEHAB of the present invention is useful as a reagent in compositions, methods and kits for the detection, treatment, and diagnosis of primary CNS tumors, including, but not limited to immunotherapy of primary CNS tumors, such as glioma.

Glycosylation-variant BEHAB can be made according to the methods disclosed herein. That is, the present invention comprises methods for the isolation of glycosylation-variant BEHAB from the particulate fraction of brain homogenate, and further includes methods for the differentiation of glycosylation-variant BEHAB from other BEHAB molecules, including full-length BEHAB and GPI-linked BEHAB.

The present invention further comprises methods for the generation of glycosylation-variant BEHAB in a recombinant cell. That is, the skilled artisan, when equipped with the present disclosure and the data herein, can produce glycosylation-variant BEHAB by transfecting a cell with an isolated nucleic acid encoding BEHAB, or a fragment thereof, and isolating glycosylation-variant BEHAB from a cell. Isolated nucleic acids for this purpose are disclosed elsewhere herein, as are methods for the transfection and expression of a protein in a cell. Preferably, the cell is a cell that expresses glycosylation-variant BEHAB, such as, but not limited to, an Oli-neu and a U87-MG cell.

As described by the data disclosed herein, a glycosylation-variant BEHAB and a poly-sialyated BEHAB can be differentiated from full-length BEHAB or GPI-anchored BEHAB through various methods. Such methods include SDS-PAGE electrophoresis, immunofluorescence and localization, immunoprecipitation, and the like. Further, the skilled artisan would readily be able to distinguish between a different isoform of a protein based on glycosylation using techniques known in the art and described herein.

VII. Methods

A. Methods of Treating a Primary CNS Tumor

The present invention is based, in part, on the novel discovery that BEHAB plays a significant role in primary CNS tumor progression, invasiveness and the survival time of mammals with brain tumors. The present invention includes a method of treating a primary CNS tumor in a mammal, preferably a human.

The present invention comprises a method of treating a primary CNS tumor or reactive gliosis in a mammal, including a human, by administering to the mammal an effective amount of glycosylation-variant BEHAB isoform inhibitor. That is, the present invention encompasses a method for treating a primary CNS tumor in a mammal, including, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like. The method comprises administering an antibody to a mammal wherein the antibody or other ligand binds to a glycosylation-variant BEHAB isoform and thus treats a primary CNS tumor. This is because, as demonstrated by the data disclosed herein, glycosylation-variant BEHAB is the major isoform of BEHAB present in primary CNS tumors, including gliomas and the like. Therefore, the present invention is useful in inhibiting the activity of a glycosylation-variant BEHAB in the CNS and thus treating a primary CNS tumor.

Methods for the generation and administration of an antibody that specifically binds a glycosylation-variant BEHAB isoform are well known in the art and are described elsewhere herein. The present invention further comprises intrabodies, antibodies administered as a protein, and antibodies administered as a nucleic acid construct encoding an antibody that binds a glycosylation-variant BEHAB isoform, including poly-sialyated BEHAB, an underglycosylated BEHAB isoform and an unglycosylated BEHAB isoform.

B. Methods of Diagnosing a Primary CNS Tumor

The present invention further encompasses methods for the diagnosis of primary CNS tumors, other central nervous system tumors, and other neuropathological disorders relating to BEHAB, including, but not limited to, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like. This is because, as demonstrated by the data disclosed elsewhere herein, expression of glycosylation-variant BEHAB and poly-sialyated BEHAB is specific to malignant gliomas, and is not present in other neurolgical pathologies nor is it present in benign tumors. The present invention therefore includes methods of detecting the expression of glycosylation-variant BEHAB or poly-sialyated BEHAB in a mammal, and therefore a method of diagnosing a primary CNS tumor and a method of differentially diagnosing a benign glioma from a malignant glioma. In all instances recited herein, whether treating or diagnosing a primary CNS tumor, the most preferred mammal is a human.

The invention includes a method of diagnosing a malignant glioma in a mammal. The method comprises obtaining a biological sample from a mammal and detecting the presence of glycosylation-variant BEHAB in that sample. Detectable levels of glycosylation-variant BEHAB, as demonstrated by the data disclosed herein, is a specific diagnostic marker of a malignant glioma, such as a grade III or grade IV glioma.

The invention also encompasses a method of differentially diagnosing a malignant glioma in a mammal, including a human, in vivo or in vitro. That is, the present invention includes a method of differentially diagnosing a glioma either in a mammal or in a biological sample from a mammal. The method further allows for the differential diagnosis between a malignant or high-grade glioma and a benign or low grade glioma. The method comprises detecting the expression of glycosylation-variant BEHAB in a mammal suspected of having a glioma. The presence of detectable glycosylation-variant is a indication that the mammal has a malignant or high grade glioma.

A malignant or high grade glioma can include, but is not limited to, a glioma, glioblastoma multiforme, anaplastic astrocytoma, diffuse astrocytoma, oligodendroglioma, and glioma subtypes grades II-IV. A low grade or benign glioma can include, but is not limited to an oligodendroglioma associated with chronic epilepsy, astrocytoma associated with chronic epilepsy, ependymoma, pilocytic astrocytoma and pleomorphic xantoastrocytoma.

Comparing the level of glycosylation-variant BEHAB in a biological sample can be accomplished using any of the methods disclosed herein or known in the art, including detection with an antibody, such as ELISA, immunoblotting techniques, protein detection techniques, such as SDS-PAGE electrophoresis, and other techniques well known in the art. As an example, a biological sample can be obtained from a mammal, and assessed for the presence of glycosylation-variant BEHAB in that sample. The biological sample can include, but is not limited to, blood, urine, feces, neural tissue, cerebrospinal fluid, saliva, brain tissue, and the like. The biological sample can be obtained by various methods depending on the biological sample to be obtained. For example, blood can be obtained through venipuncture; urine, feces, and saliva can be captured in a specimen vessel and the like. Tissue samples, including, but not limited to brain tissue and neural tissue can be obtained through a biopsy or similar methods well known in the art. Cerebrospinal fluid can be collected through a spinal tap using methods well known in the art.

For the in vivo detection of glycosylation-variant BEHAB or the diagnosis of a glioma related to glycosylation-variant BEHAB, the skilled artisan can employ a tagged antibody for the detection of glycosylation-variant BEHAB in a mammal. Such antibodies can be generated using techniques described elsewhere herein and then conjugated to a tag or other molecule capable of detection through a number of methods. Methods of conjugating a tag or other molecule to an antibody are well known in the art and can be accomplished using techniques in protein chemistry, described elsewhere herein. As an example, an antibody that binds glycosylation-variant BEHAB can be conjugated to a radioactive isotope and the binding of the isotope tagged antibody can be detected on a film sensitive to radioactivity, such as X-ray film. The antibody can also be bound to a tag visible to magnetic resonance imaging technology. Further, the present invention includes a method in which an antibody is conjugated to fluorescent molecule, such as luciferase or green fluorescent protein, or another tag, such as horseradish-peroxidase, a fluorescent molecule, an enzyme, gold, biotin, a radioactive isotope, or gadolinium, and the binding of the antibody to a glycosylation-variant BEHAB isoform is detected through an imaging system capable of visualizing a tag. Uses of biophotonic imaging systems for the in vivo detection of fluorescent tags are well known in the art and such systems are available commercially (Xenogen, Alameda, Calif.).

The invention further includes a method of diagnosing primary CNS tumor progression in a mammal. As will be appreciated by the skilled artisan, once armed with the present disclosure and the data herein, detectable glycosylation-variant BEHAB is specific for malignant tumors. Therefore, the present invention includes a method of diagnosing brain tumor progression in a mammal. The method comprises obtaining a biological sample from a mammal and detecting the presence of glycosylation-variant BEHAB in that sample. The presence of glycosylation-variant BEHAB in the sample can then be compared to samples obtained earlier or later from the same mammal, including a human, in order to determine the expression of glycosylation-variant BEHAB in earlier or later samples. A lesser detectable level or no detectable level of glycosylation-variant BEHAB in the sample indicates that the mammal is in regression or the anti-tumor treatment administered to the animal is effective. A higher detectable level of glycosylation-variant BEHAB indicates that the tumor is progressing and that other courses of therapy should be used. This is because, as disclosed elsewhere herein, a detectable level of glycosylation-variant BEHAB in a mammal is specific for a malignant or high-grade glioma.

One of skill in the art will appreciate, when armed with the present disclosure and data herein, that methods for determining the level of glycosylation-variant BEHAB cleavage include, but are not limited to Western blotting, ELISA, and other immuno-detection assays well known in the art.

In one aspect, the biological sample is selected from the group consisting of a blood sample, a neurological tissue biopsy, a cerebrospinal fluid sample, urine, saliva, and the like.

The invention includes a method of assessing the effectiveness of a treatment for a primary CNS tumor in a mammal. The method comprises assessing the level of glycosylation-variant BEHAB expression, amount, and/or activity, before, during and after a specified course of treatment for a disease, disorder or condition mediated by or associated with increased BEHAB expression (e.g., a malignant glioma). This is because, as stated previously elsewhere herein, increased glycosylation-variant BEHAB expression, amount and/or activity is associated with or mediates the malignancy of a glioma. Thus, assessing the effect of a course of treatment upon glycosylation-variant BEHAB expression/amount/activity indicates the efficacy of the treatment such that a lower level of glycosylation-variant BEHAB expression, amount, or activity indicates that the treatment method is successful.

The course of therapy to be assessed can include, but is not limited to, surgery, chemotherapy, radiation therapy, and/or the multiple modes of therapy for a glioma disclosed herein.

C. Methods of Identifying Useful Compounds

The present invention further includes a method of identifying a compound that affects expression of glycosylation-variant BEHAB isoform and/or a poly-sialyated BEHAB isoform, in a cell. The method comprises contacting a cell with a test compound and comparing the level of expression of glycosylation-variant BEHAB and/or a poly-sialyated BEHAB in the cell so contacted with the level of expression of glycosylation-variant BEHAB and/or a poly-sialyated BEHAB in an otherwise identical cell not contacted with the compound. If the level of expression of glycosylation-variant BEHAB and/or a poly-sialyated BEHAB is higher or lower in the cell contacted with the test compound compared to the level of expression of glycosylation-variant BEHAB and/or a poly-sialyated BEHAB in the otherwise identical cell not contacted with the test compound, this is an indication that the test compound affects expression of glycosylation-variant BEHAB and/or a poly-sialyated BEHAB in a cell.

The invention encompasses methods to identify a compound that affects expression of glycosylation-variant BEHAB and/or a poly-sialyated BEHAB. One skilled in the art would appreciate, based upon the disclosure provided herein, that assessing the level of glycosylation-variant BEHAB and/or a poly-sialyated BEHAB can be performed using probes (e.g., antibodies that specifically bind with glycosylation-variant BEHAB and/or a poly-sialyated BEHAB), such that the method can identify a compound that selectively affects expression of BEHAB isoforms. Such compounds are useful for inhibiting expression of glycosylation-variant BEHAB and/or a poly-sialyated BEHAB. One skilled in the art would understand that such compounds can be useful for inhibiting a disease, disorder, or condition mediated by and/or associated with increased expression of BEHAB isoforms, e.g., the presence of glycosylation-variant BEHAB and/or a poly-sialyated BEHAB is associated with malignant gliomas.

Similarly, the present invention includes a method of identifying a compound that reduces expression of glycosylation-variant BEHAB in a cell. The method comprises contacting a cell with a test compound and comparing the level of expression of glycosylation-variant BEHAB in the cell contacted with the compound with the level of expression of glycosylation-variant BEHAB in an otherwise identical cell, which is not contacted with the compound. If the level of expression of glycosylation-variant BEHAB is lower in the cell contacted with the compound compared to the level in the cell that was not contacted with the compound, then that is an indication that the test compound reduces expression of glycosylation-variant BEHAB in a cell.

The skilled artisan will further appreciate that the present invention is not limited to a method of identifying a useful compound in a cell or an animal. That is, the present invention includes methods of identifying a useful compound in a cell-free system. A cell-free system, as used herein, refers to an in vitro assay wherein the components necessary for a reaction to take place are present, but are not associated with a cell. Such components can include cellular enzymes, transcription factors, proteins, antibodies, nucleic acids, and the like, provided that they are substantially free from a cell. Detecting glycosylation-variant BEHAB assays can be performed free of a cell or animal, including the use of immunoprecipitation assays and the like. Thereby, the present invention includes a method of identifying a useful compound for treating a glioma in a cell-free system.

VIII. Kits

The present invention encompasses various kits which comprise a compound, including an antibody that specifically binds glycosylation-variant BEHAB, an antibody that specifically binds poly-sialyated BEHAB, an applicator, and instructional materials which describe use of the compound to perform the methods of the invention. Although model kits are described below, the contents of other useful kits will be apparent to the skilled artisan in light of the present disclosure. Each of these kits is contemplated within the present invention.

The present invention comprises a kit for detecting a glycosylation-variant BEHAB isoform. The kit comprises an antibody to a glycosylation-variant BEHAB isoform. Such antibodies are disclosed are set forth elsewhere herein. The kit further comprises an instructional material comprising information on how to use the antibody for the detection of a glycosylation-variant BEHAB isoform, including instructions to accomplish the methods set forth elsewhere herein.

The present invention further comprises a kit for diagnosing a malignant glioma in a mammal. The kit comprises an antibody that specifically binds a glycosylation-variant BEHAB isoform, an applicator and a instructional method for the use of the kit. Uses of an applicator and methods for the diagnosis of a malignant glioma are disclosed elsewhere herein.

The present invention further comprises a kit for diagnosing a malignant glioma in a mammal. The kit comprises an antibody that specifically binds a poly-sialyated BEHAB isoform, an applicator and a instructional method for the use of the kit. Uses of an applicator and methods for the diagnosis of a malignant glioma are disclosed elsewhere herein.

The invention also includes a kit for treating a malignant glioma. The kit includes a composition comprising an antibody that specifically binds a glycosylation-variant BEHAB isoform, or a fragment thereof, a pharmaceutically acceptable carrier, and an applicator. Methods for using an antibody and applicator are set forth elsewhere herein. The instructional material comprises the methods disclosed herein for the treatment of a malignant glioma.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

The materials and methods used in the experiments presented in this Example are now described.

Human Tissue:

All studies regarding samples of human tissue were performed in compliance with the guidelines of the Human Investigations Committee at Yale University School of Medicine. Pathologically graded fresh-frozen surgical samples of intracranial tumors (20 male, 12 female, ages 13-64 years), including glioma, meningioma, epidermoid tumor, schwannoma and medulloblastoma were obtained from Yale-New Haven Medical Hospital (New Haven, Conn.). Human glioma samples (8 female, 13 male) were independently graded as previously described (Jaworski et al., 1996, Cancer Res, 56: 2293-2298). Postmortem brain samples (10 female, 10 male, with postmortem interval ranging from 2 to 31 hours) from individuals who had died without neurological pathologies or complications served as controls for the normal level of BEHAB protein expression in normal human cortex. Samples of normal temporal and parietal human brain cortex (10 male, 10 female, ages 16 gestational weeks to 76 years) were obtained from the Brain and Tissue Banks for Developmental Disorders (University of Maryland, Baltimore Md.). Fresh-frozen surgical samples of epilepsy foci (2 male, 1 female, ages 9-50 years) were kindly provided by Dr. D. Spencer (Department of Neurosurgery, Yale University Medical School). Postmortem brain cortex samples (3 male, 1 female, ages 78-87 years) from individuals diagnosed with Alzheimer's disease were kindly provided by Dr. G. W. Rebeck (Department of Neuroscience, Georgetown University, Washington D.C.). All samples were stored at −70° C. until further processing.

Subcellular Fractionation:

Brain and tumor samples were quickly thawed on ice and homogenized in 10 volumes of 25 mM Tris HCl, pH 7.4, containing 0.32 M sucrose (TS buffer) containing a protease inhibitor cocktail (Complete, EDTA-free, Roche, Nutley, N.J.). The homogenate was centrifuged at 950 g×10 minutes and the nuclear pellet (P1) was washed once by rapid rehomogenization in TS buffer and centrifuged as above. Post-nuclear supernatants were combined and centrifuged at 100,000 g×60 minutes to provide total particulate (membrane-enriched) and soluble fractions. Aliquots of the subcellular fractions were equilibrated at a final total protein concentration of 1-2 mg/ml in CH buffer (40 mM Tris HCl, 40 mM sodium acetate, pH 8.0), containing 5 mM EDTA, and treated with 0.25 U/ml of protease-free chondroitinase ABC from Proteus vulgaris (EC 4.2.2.4, Seikagaku, East Fallmouth, Mass.) for 8 hours at 37° C. Chondroitinase activity was stopped by boiling the samples in the presence of 1× gel-loading buffer. Samples (10-15 μg total protein) were electrophoresed on reducing 6% SDS-polyacrylamide gels and analyzed by Western blotting and semiquantitative densitometry.

Release of BEHAB/Brevican Isoforms from Brain Membranes:

To characterize the association of different BEHAB isoforms with the cell membrane, total membranes (˜1 mg total protein/ml) obtained from control and glioma samples were resuspended in 50 mM Tris HCl buffer, pH 7.4, in the presence or absence of 10 mM EDTA for 1 hour at 4° C. Alternatively, membranes were resuspended in 100 mM sodium carbonate, pH 11.3, for 30 minutes at 4° C. After incubation, membranes were centrifuged at 20,800 g for 20 minutes. Released BEHAB was recovered in the supernatant, and the membranes containing retained BEHAB were washed twice with 50 mM Tris HCl buffer and resuspended in the same initial volume. All samples were finally equilibrated with CH buffer and treated with chondroitinase ABC prior to protein electrophoresis. For immunoprecipitation studies, membranes were first extracted for 1 hour at 4° C. in 50 mM Tris HCl, pH 7.4, containing 300 mM NaCl and 0.6% w/v CHAPS (3-[(3-chloamidopropyl)dimethylammonio]-1-propanesulfonic acid. Solubilized proteins were immunoprecipitated with the rabbit polyclonal anti-BEHAB antibody B6, described elsewhere herein, preadsorbed to protein A-sepharose (Amersham-Pharmacia Biotech, Piscataway, N.J.), according to standard protocols known in the art.

Cell Cultures and Transfections:

The human glioma cell line U87-MG (American Type Culture Collection, Manassas, Va.) was grown at 5% CO₂ in DMEM medium (Gibco, Gaithersburg, Md.) supplemented with 10% fetal calf serum (FCS) (Hyclone, Logan Utah), 50 μg/ml penicillin and 50 μg/ml streptomycin (Gibco, Gaithersburg, Md.). A clone comprising the complete coding sequence of human BEHAB (GenBank Accession No. BC010571, nucleotides 1-3245) was purchased from Invitrogen (La Jolla, Calif.) and subcloned from the original pSPORT6.1 plasmid into the EcoRI-NotI restriction sites of a pcDNA3.1(+) plasmid and a pcDNA3.1-V5(6×His) plasmid (Invitrogen, La Jolla, Calif.). Cells were transfected employing Lipofectamine 2000 (Invitrogen, La Jolla, Calif.) at a ratio of Lipofectamine (μl):DNA (μg) of 2:1 according to the manufacturers protocol. Control transfections were performed with the parental pcDNA3.1 (+) vector.

Preparation of Cell Membranes and Immunocytochemistry:

Transfected cells were changed to serum-free medium Optimem (Gibco, Gaithersburg, Md.) 24 hours post-transfection and collected 24 hours after the medium change. Collected cells were lysed in 25 mM phosphate buffer, pH 7.4, containing a protease inhibitor cocktail (Complete, EDTA-free, Roche, Nutley, N.J.) and 2 U/ml RNAse-free DNAse I (Roche, Nutley, N.J.). Total membranes were obtained by centrifugation at 20,800 g×30 minutes and prepared for protein electrophoresis. Culture medium was concentrated by ultradiafiltration and equally processed for SDS-PAGE.

For live immunocytochemical staining of transfected U87-MG cells, cultures were grown on poly-L-lysine (100 μg/ml, Sigma, St. Louis, Mo.) coated 18 mm glass coverslips in 12-well plates for 24 hours before transfection with human BEHAB cDNA. Unfixed, unpermeabilized cultures were repeatedly rinsed in DMEM and incubated with the rabbit polyclonal anti-BEHAB antibody B6 or anti-V5 antibody at 4° C. for 30 minutes before fixation. Cells were subsequently rinsed and then fixed for 20 minutes in 4% paraformaldehyde in 100 mM phosphate buffer, pH 7.4, incubated for 60 minutes with Alexa-conjugated anti-rabbit IgG secondary antibodies (Molecular Probes, Eugene, Oreg.), briefly counter-stained with DAPI (0.25 μg/ml, Sigma, St. Louis, Mo.) and prepared for fluorescence microscopy.

To determine which isoform(s) of BEHAB had been detected in the cell surface by the live-cell staining procedure, transfected U87-MG cells were rinsed in DMEM, incubated with control medium at 4° C. for 30 minutes and scraped from the wells just before the fixation step. These cells were homogenized in 25 mM phosphate buffer, pH 7.4, and the total homogenates were prepared for protein electrophoresis.

Protein Deglycosylation:

Soluble and particulate fractions from control brain and glioma samples were equilibrated in deglycosylation buffer (20 mM Tris HCl, 20 mM sodium acetate, 25 mM NaCl, pH 7.0) at a protein concentration of 1 mg/ml, and treated with the following glycosidases alone or in combination: 0.25 U/ml chondroitinase ABC, 20 mU/ml O-glycosidase from Diplococcus pneumoniae (EC 3.2.1.97, Roche, Nutley, N.J.), 100 mU/ml sialidase from Arthrobacter ureafaciens (EC 3.2.1.18, Roche, Nutley, N.J.) and 100 U/ml glycopeptidase F (PNGase F) from Chryseobacterium meningosepticum (EC 3.5.1.52, Calbiochem, La Jolla, Calif.). In all cases samples were incubated with the enzymes for 8 hours at 37° C. in the presence of protease inhibitors. Enzyme digestions were stopped by boiling the samples in 1× gel-loading buffer.

For denaturing deglycosylation, required for non-exposed N-linked carbohydrates, samples were first equilibrated in 0.1% w/v SDS and 0.1 M 2-mercaptoethanol and heated at 95° C. for 10 minutes. Subsequently, samples were equilibrated in deglycosylation buffer containing 0.8% v/v Nonidet-P40 and deglycosylation proceeded in the same conditions as above.

Western Blot Analysis:

Samples (10-15 μg total protein) were electrophoresed on reducing 6% SDS-polyacrylamide gels and proteins were electrophoretically transferred to nitrocellulose. Blots were incubated with an affinity-purified rabbit polyclonal antibody (B6) produced against a synthetic peptide corresponding to the chondroitin sulfate attachment region (amino acids 506-529) of rat BEHAB. Alternatively, BEHAB was detected with affinity-purified rabbit polyclonal antibodies produced against synthetic peptides corresponding to the amino acids 60-73 of rat BEHAB (antibody B5) and the amino acids 859-879 of human BEHAB (antibody B_{CRP}). The antibodies B6, B5 and BCRP were previously described for the specific detection or BEHAB in rat brain samples (Matthews et al, 2000, J. Biol. Chem. 275: 22695-22703; Viapiano et al., 2003, J. Biol. Chem. 278: 33239-3347). The N-terminal cleavage product of BEHAB was detected with a rabbit polyclonal antibody against the cleavage neoepitope QEAVESE (SEQ ID NO: 9; amino acids 389-395 of rat BEHAB. V5 tagged human BEHAB was also detected using a mouse monoclonal anti-V5 antibody (Invitrogen, La Jolla, Calif.). Alkaline-phosphatase conjugated secondary antibodies were employed and the immunoreactive bands were visualized with nitro-blue tetrazolium and 5-bromo-4-chloro-3-indoyl phosphate. For densitometric quantification of BEHAB in total homogenates, immunoreactive bands were visualized by chemiluminiscence (Amersham, Piscataway, N.J.) and quantified using the Gel-Pro v3.1 software (Media Cybernetics, Silver Spring, Md.). Statistical comparisons were performed by Student's t-test with Welch's correction for non-homocedacy.

The results of the experiments presented in this Example are now described.

Gliomas are able to infiltrate into the surrounding normal neural tissue, which is a characteristic that is quite unique and distinct to these tumors. In order for glioma cells to disperse into normal neural tissue, they need to navigate through the unique extracellular environment found in the CNS. Therefore, molecules uniquely expressed in glioma cells that modify their interaction with the neural environment are of particular interest. Disclosed herein are tumor-specific isoforms of the CNS-specific ECM component BEHAB in human gliomas. The unique expression profile of BEHAB in these tumors demonstrates an important role for this glycoprotein in glioma.

BEHAB mRNA is expressed at appreciable levels in normal brain and significantly upregulated in malignant gliomas (Gary et al., 2000, Gene 256: 139-147; Boon et al., 2002, Proc. Nat'l Acad. Sci. USA 99: 11287-11292). The presence of BEHAB protein in normal brain and its upregulation in glioma is demonstrated herein. However, these results indicate that the upregulation in glioma leads not only to a general increase in the expression of BEHAB but also to the glioma-specific expression of differentially glycosylated isoforms, poly-sialyated BEHAB and glycosylation-variant BEHAB. While the over-expression of many proteins has been described in glioma, the expression of tumor-specific proteins or protein isoforms is relatively rare, demonstrating the therapeutic and diagnostic value for the BEHAB isoforms described here.

BEHAB is Differentially Expressed in Normal Human Brain and Primary Brain Tumors

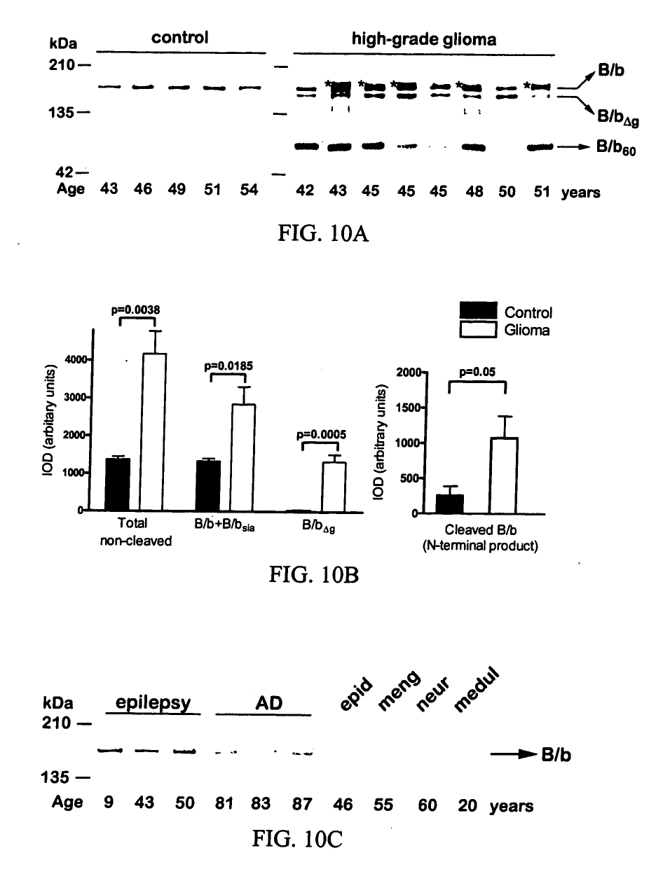

The expression of BEHAB protein in normal brain and glioma tissue was analyzed by Western blot after subcellular fractionation and enzymatic removal of chondroitin sulfate chains. In normal brain tissue, secreted BEHAB was detected as an approximately 160-kDa full-length form, as well as cleavage products of approximately 60 and approximately 100-kDa (FIG. 9) generated by specific proteolysis by the disintegrin and metalloprotease with thrombospondin motifs (ADAMTS)-4/Aggrecanase-1 (Matthews et al., 2000, J. Biol. Chem. 275: 22695-22703; Nakamura et al., 2000, J. Biol. Chem. 275: 38885-38890). In surgical samples from human gliomas there was an expected increase in the intensity of the same protein bands, since previous work demonstrated that BEHAB mRNA expression is dramatically upregulated in glioma (Gary et al., 2000, Gene 256: 139-147; Jaworski et al., 1996; Cancer Res. 56: 2293-2298). However, the protein analysis disclosed a more complex picture, revealing not only increased expression of the full-length and cleaved forms of BEHAB but also the presence of additional, unique isoforms specific to glioma. The most evident isoform, glycosylation-variant BEHAB, migrated at an apparent molecular mass of ˜150-kDa and distributed exclusively to membrane-containing fractions. A second, less conspicuous isoform, poly-sialyated BEHAB, migrated at a slightly higher apparent molecular mass than the ˜160-kDa form and was distributed both in soluble and particulate fractions of glioma samples.

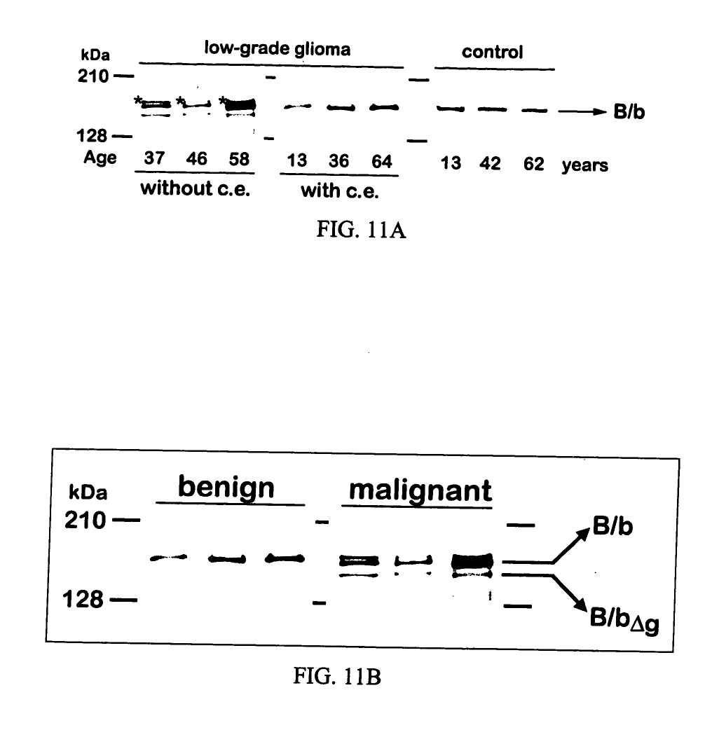

Glycosylation-variant BEHAB was present in every sample of high-grade glioma, grades III (n=3) and IV (n=19), assayed to date (FIG. 10A), while poly-sialyated BEHAB appeared in about half of the high-grade gliomas analyzed. Densitometric analysis of the expression of these isoforms demonstrated that total expression of all full-length BEHAB isoforms was over 3-fold higher in gliomas compared to normal brain tissue. This increase in BEHAB expression was similarly reflected by an approximately 4-fold increase of the 60-kDa cleavage product in gliomas versus normal tissue. Cleaved full-length BEHAB was independently quantified from Western blots of the N-terminal cleavage product. The expression of both full-length and cleaved BEHAB were significantly increased in gliomas compared to controls (Student's t-test) (FIG. 10B). Remarkably, the expression of glycosylation-variant BEHAB alone accounted for roughly half of the total overexpression above control levels for non-cleaved BEHAB, suggesting that a substantial proportion of BEHAB synthesized in glioma is shunted to the pathway that makes this isoform.

To determine if poly-sialyated BEHAB and glycosylation-variant BEHAB were unique to gliomas or were also expressed in other neuropathologic conditions, samples from other types of intracranial tumors (n=4) as well as brain cortex from epilepsy (n=3) and Alzheimer's disease (n=4) cases were analyzed. None of these samples had detectable levels of the glioma-specific isoforms (FIG. 10C).

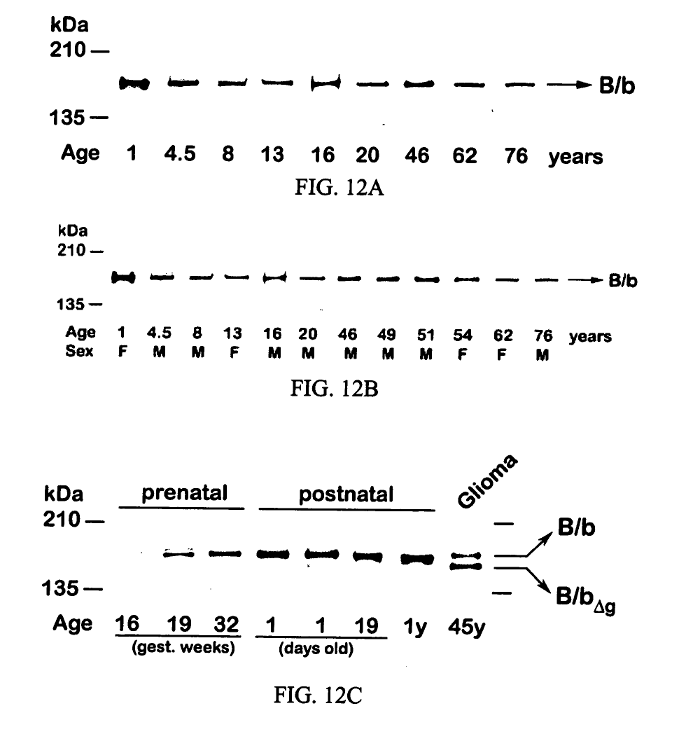

Low-grade gliomas are a heterogeneous group of diseases characterized by relatively slow-growing primary brain tumors of astrocytic and/or oligodendroglial origin. Many patients present with easily controlled seizures and remain stable for years, whereas others progress rapidly to higher-grade tumors. There are few good molecular or histological prognostic markers for low-grade glioma, despite the fact that clinical outcomes for patients with these tumors vary widely. There is a pressing need for assays that can discriminate between benign and malignant tumors. The histology of oligodendrogliomas does not often predict clinical behavior. For example, some oligodendrogliomas have an indolent course over more than a decade; others behave in a malignant fashion, progressing over a few years. The data disclosed herein demonstrates that a set of oligodendrogliomas, known to be benign, do not express glycosylation-variant BEHAB, whereas those that behave in malignant fashion do express glycosylation-variant BEHAB (FIG. 11). While prognostic markers for high-grade oligodendrogliomas have been found, at present, there are no molecular markers that distinguish these low-grade tumors from each other. Therefore, the expression of glycosylation-variant BEHAB represents an important diagnostic method to distinguish benign low-grade tumors from malignant tumors.

Because of the unique expression of poly-sialyated BEHAB and glycosylation-variant BEHAB in high-grade gliomas, the correlation between the expression of BEHAB isoforms and tumor grade was investigated. Analysis of grade II oligodendrogliomas (n=6) revealed two subsets of samples, one expressing both poly-sialyated BEHAB and glycosylation-variant BEHAB and the other one not expressing either of these isoforms (FIG. 11). The subset of samples that were negative for both poly-sialyated BEHAB and glycosylation-variant BEHAB corresponded to patients diagnosed with low-grade tumors associated with chronic epilepsy, a subclass of gliomas that are notably indolent and benign (Bartolomei et al., 1997 J. Neurooncol. 34: 79-84; Luyken et al., 2003, Epilepsia 44: 822-830). As disclosed elsewhere herein, the expression profile of BEHAB isoforms in these tumors represents the first known molecular marker that distinguishes these benign tumors from other low-grade gliomas.

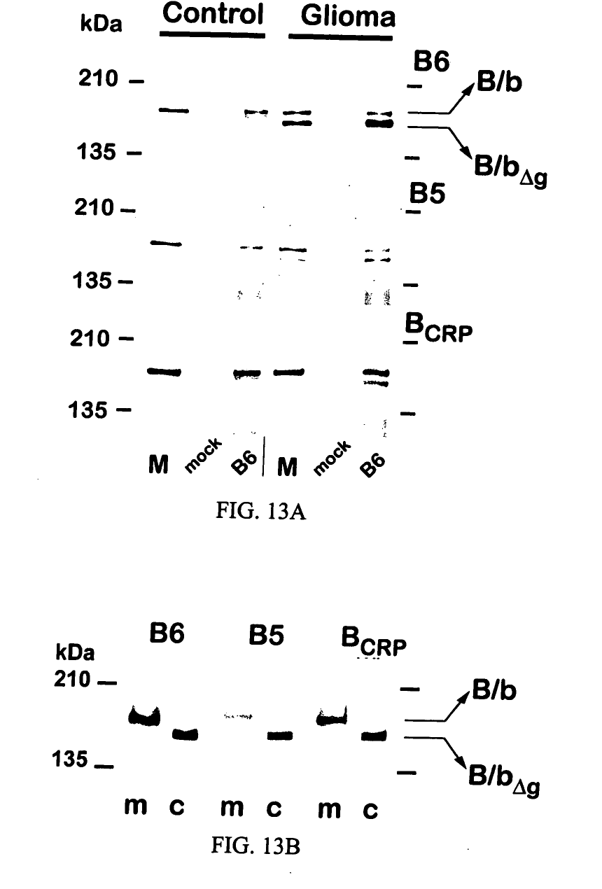

Rat BEHAB is expressed in a developmentally-regulated manner (Viapiano et al., 2003, J. Biol. Chem. 278: 33239-33247). The ontogenetic expression of the glioma-specific isoforms described here were analyzed to determine whether they could be detected during normal brain development. None of the samples from individuals older than 1 year of age (n=14, FIG. 12A) contained detectable amounts of either poly-sialyated BEHAB or glycosylation-variant BEHAB. Only the ˜160-kDa isoform of BEHAB was detected, which was expressed at its highest levels before 8 years of age and declined afterwards to a lower and constant level throughout adulthood. However, at earlier developmental ages (n=6), from 16 weeks of gestation to 19-day-old infants, a faint band migrating at the position of glycosylation-variant BEHAB was observed (FIG. 12B). This band was barely detectable in total homogenates from cortical tissue but was clearly observed in the membrane-enriched fraction.

Poly-sialyated BEHAB is present in roughly half of all the high- and low-grade gliomas analyzed, an incidence similar to that of the tumor-specific variant of the EGF receptor, EGFR vIII, which is the most typical cell-surface marker for high-grade gliomas (Kleihues et al., 2000, Int'l. Agency for Research on Cancer, IARC Press, London). Apart from the differential glycosylation of poly-sialyated BEHAB, which includes additional sialic acid on O-linked carbohydrates, all other biochemical properties of this isoform, including subcellular distribution and membrane attachment seem to be identical to the normal secreted 160-kDa full-length isoform of BEHAB. The presence of abnormally sialylated cell surface glycoproteins is a typical modification in several tumors, including gliomas, associated with malignant behavior (Hakamori, 2001, Adv. Exp. Med. Biol. 491: 369-402; Kim and Varki, 1997, Glycoconj. J. 14: 569-576; Yamamoto et al., 1997, Brain Res. 755: 175-179). BEHAB represents a novel substrate for over-sialylation, indicating the importance of BEHAB function in gliomas as well as association with clinical outcome.

The most conspicuous glioma-specific isoform of BEHAB, glycosylation-variant BEHAB, is a full-length product of BEHAB mRNA that arises from an incomplete or reduced glycosylation of the core protein. Glycosylation-variant BEHAB is absent from the normal adult brain but was found in every sample of high-grade gliomas analyzed to date is thus a novel glioma-specific marker in adult human brain. Glycosylation-variant BEHAB is only absent in a restricted subset of low-grade oligodendrogliomas that have been characterized as a unique pathological entity among primary brain tumors (Bartolomei et al., 1997, J. Neurooncol. 491: 79-84; Luyken et al., 2003, Epilepsia 822-830). These low-grade, epileptogenic oligodendrogliomas have a predominant cortical localization and uniquely benign pathological features. A proper identification of this particular subtype of tumors is critical for establishing prospective survival and directing therapy. At present, these tumors cannot be distinguished by histology or chromosomal (i.e. 1p/19q) features from typical low-grade oligodendrogliomas, which have a more aggressive profile and require a distinct clinical approach. Glycosylation-variant BEHAB is the first molecular marker that distinguishes between these indolent tumors from more aggressive low-grade gliomas, therefore demonstrating diagnostic utility.

Glioma-specific poly-sialyated BEHAB and glycosylation-variant BEHAB isoforms are absent not only in the normal adult human brain but also in other neuropathologies such as Alzheimer's disease, epilepsy and several non-glial intracranial tumors. Therefore, their appearance is not likely to reflect a general pathogenic or gliotic process but, instead, is a result of modifications specific to gliomas. Only glycosylation-variant BEHAB is expressed at very low levels during the second half of prenatal and first days of postnatal development, a period of intense gliogenesis (Kadhim et al., 1988, J. Neuropathol. Exp. Neurol. 47: 166-188; Marin-Padilla, 1995, J. Comp. Neurol. 357: 554-572), and disappears by the first year of age. Expression of glycosylation-variant BEHAB in gliomas represents a re-activation of early developmental programs, a mechanism that has previously been implicated in glioma progression (Seyfried, 2001, Perspect. Biol. Med. 44: 263-282).

Glioma-Specific Glycosylation-Variant BEHAB is a Full-Length Isoform of BEHAB

In the rodent brain several isoforms of BEHAB smaller than the full-length secreted protein are generated by alternative splicing, specific proteolytic processing and differential glycosylation, while larger than full-length BEHAB isoforms are only produced by additional glycosylation with chondroitin sulfate chains. The mechanisms involved in the production of the glioma-specific isoforms of BEHAB were investigated.

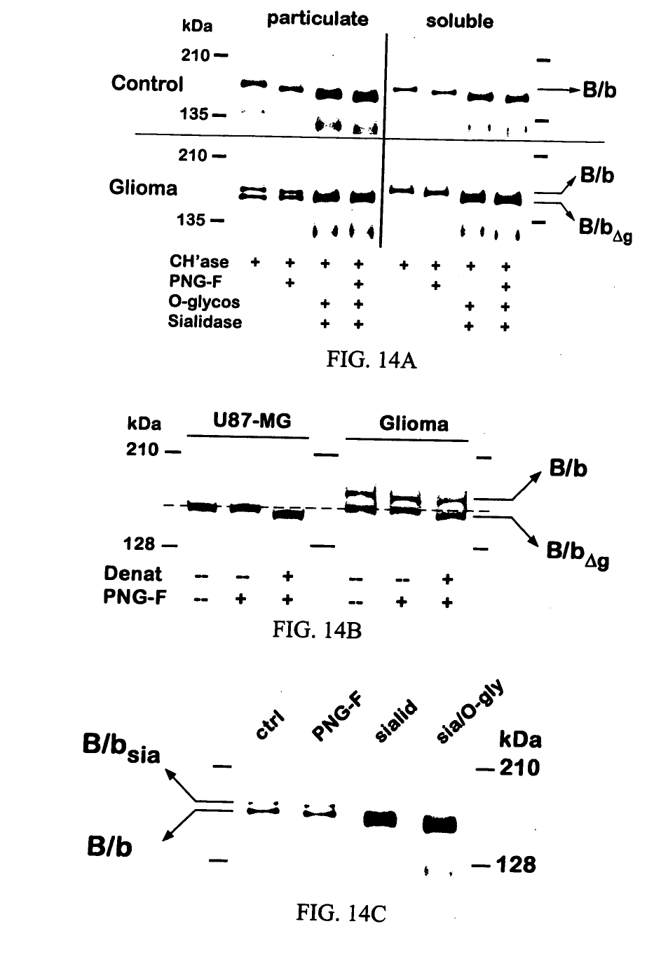

The amino acid sequence of BEHAB is incomplete in proteolytic products and in a splice variant that lacks the C-terminal globular domain. The antibodies B5 and BCRP, directed against epitopes located at less than 5 kDa from the N- and C-termini of the full-length protein, respectively (see FIG. 9A), were used to determine if the glycosylation-variant BEHAB isoform contained the complete amino acid sequence of full length BEHAB. BEHAB was immunoprecipitated from detergent extracts of normal brain and glioma membranes using the antibody B6 and the immunoprecipitated material was probed with the antibodies B5 and B_{CRP}. All three antibodies recognized both full-length BEHAB as well as glycosylation-variant BEHAB from glioma samples (FIG. 13A), indicating that glycosylation-variant BEHAB was neither a terminally cleaved product of full-length BEHAB nor the glycosylphosphatidylinositol-linked splice variant. Glycosylation-variant BEHAB was never detected in immunoprecipitates from control samples, which are highly enriched in full-length BEHAB, providing further evidence that glycosylation-variant BEHAB is not expressed in normal adult brain.

As a separate and complementary control to verify that glycosylation-variant BEHAB was generated from the same mRNA transcript encoding the full-length isoform of BEHAB, U87MG cells were transfected with full-length human BEHAB cDNA and the resultant expressed proteins were analyzed by Western blotting. Transfected U87MG cells produced both 160-kDa BEHAB (full-length BEHAB), secreted to the culture medium, and glycosylation-variant BEHAB, which, as in human glioma samples, localized exclusively to the particulate subcellular fraction. Poly-sialyated BEHAB was never observed in U87MG or any other of the rat and human glioma cell lines assayed.

Together, these results demonstrate that the glycosylation-variant BEHAB isoform contains the full-length peptidic sequence of BEHAB and is not produced by cleavage or alternative splicing.

Poly-Sialyated BEHAB and Glycosylation-Variant BEHAB are Generated by Differential Glycosylation of BEHAB

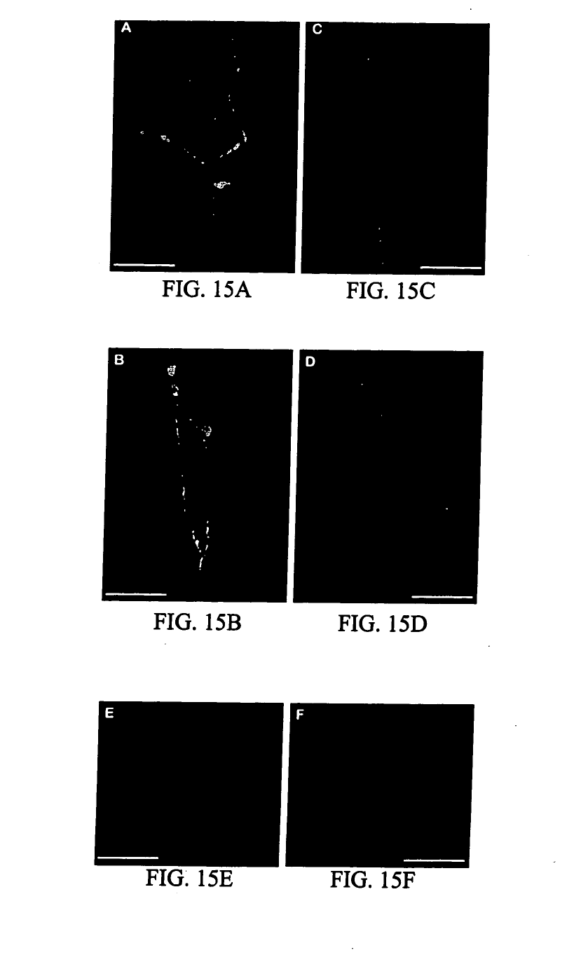

BEHAB comprises N- and O-linked oligosaccharides as well as chondroitin sulfate chains. Treatment with chondroitinase ABC produces an increased immunoreactivity of full-length BEHAB in Western blots, likely due to the electrophoretic collapse of the isoforms that carry chondroitin sulfate into a single band. Chondroitinase treatment, however, caused no effect on glycosylation-variant BEHAB mobility or immunoreactivity, demonstrating that it was glycosylated differently than full-length BEHAB. Furthermore, treatment with combinations of chondroitinase and enzymes that remove N- and O-linked sugars shifted the full-length BEHAB band towards the position of glycosylation-variant BEHAB (FIG. 14A), but did not affect the electrophoretic mobility of glycosylation-variant BEHAB, indicating that it lacked some or all the sugars present in the glycosylated, full-length isoform. Only after protein denaturation followed by deglycosylation with PNGase F was a slight change in the electrophoretic mobility of glycosylation-variant BEHAB detected (FIG. 14B). These results indicate that glycosylation-variant BEHAB carries only a few, non-exposed, N-linked carbohydrates per protein molecule, thus being an under-glycosylated form of BEHAB.

Results from deglycosylation assays also demonstrated that the higher molecular mass of the poly-sialyated BEHAB isoform was generated by increased sialic acid on O-linked carbohydrates, since both poly-sialyated BEHAB and full-length BEHAB collapsed to a single position on SDS-PAGE gels after treatment with sialidase but not with PNGase F (FIG. 14C). Together, these results demonstrate that the differences in molecular mass between the full-length BEHAB and the glioma-specific poly-sialyated BEHAB and glycosylation-variant BEHAB isoforms are due to differential glycosylation.

Glycosylation-Variant BEHAB is Expressed on the Cell Surface

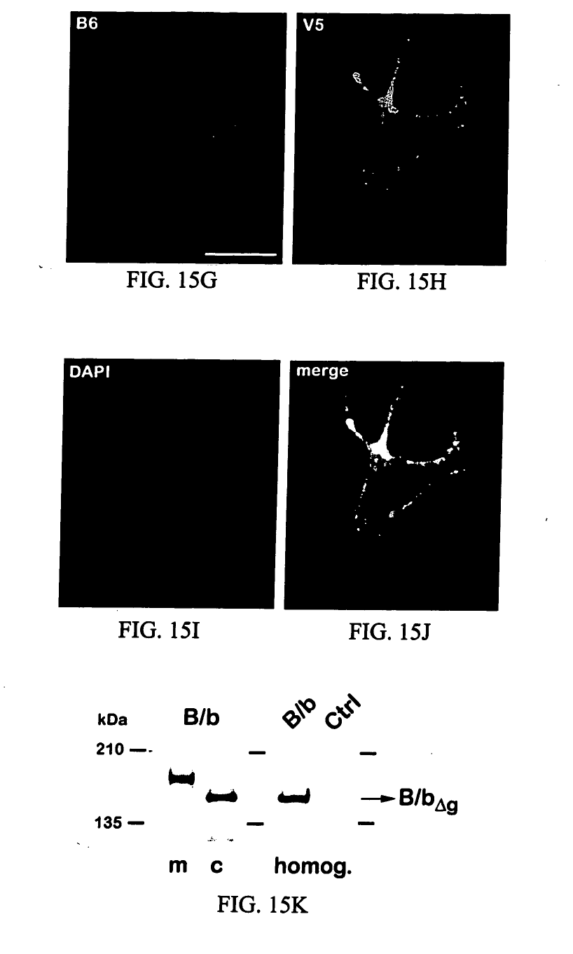

Since glycosylation-variant BEHAB is an underglycosylated form of BEHAB, a possible explanation for partitioning with the membrane fraction is that it represents a mis-folded form caused by BEHAB overexpression, which is then retained in the secretory pathway and does not reach the cell surface. To determine whether glycosylation-variant BEHAB could be localized on the extracellular surface of glioma cells, U87MG cells transfected with the cDNA for V5-tagged or untagged full-length human BEHAB were probed with B6 and anti-V5 before fixation. Results from live-cell staining, where antibodies can only detect extracellularly exposed epitopes, revealed BEHAB immunoreactivity on the extracellular surface of transfected cells (FIG. 15A-F). Further analysis of cells stained for BEHAB before and after fixation and permeabilization showed that in addition to the secretion of BEHAB to the cell surface, the protein is also found intracellularly (FIG. 15G-J). This fraction of BEHAB likely represents BEHAB progressing through the secretory pathway or an artifact of overexpression.

U87MG cells are able to make both glycosylation-variant BEHAB as well as full-length BEHAB (see FIG. 13B), and therefore it was determined if the immunoreactivity on the cell surface exclusively represented the expression of glycosylation-variant BEHAB. Transfected cells were processed exactly as described for live-cell staining but the cells were collected prior to fixation. Western blotting of total homogenates from these cells only detected glycosylation-variant BEHAB (FIG. 15H), confirming that this was the only isoform previously detected on the cell surface by immunocytochemistry.

Aberrant glycosylation of cell surface proteins occurs in almost all cancers (Hakomori, 2002, Proc. Nat'l Acad. Sci. USA 99: 10231-10233) and can disrupt normal protein-protein interactions, likely promoting tumor invasion and metastasis (Gorelik et al., 2001, Cancer Metastasis Rev. 20: 245-277; Kim and Varki, 1997, Glycoconj. J. 14: 569-576). Fully glycosylated BEHAB is invested with a diverse set of carbohydrates, including N-linked sugars, mucin-type O-linked sugars and chondroitin sulfate chains. Changes in the expression of specific glycosyltransferases or modification of metabolic pathways in glioma can be expected to produce modifications on specific carbohydrates and generate glycovariants such as poly-sialyated BEHAB. The origin of glycosylation-variant BEHAB, however, is more difficult to understand, since it is generated by a mechanism that specifically prevents the addition of carbohydrates to the protein core while other proteins are still glycosylated.

Despite being underglycosylated, glycosylation-variant BEHAB is not a precursor that accumulates in the endoplasmic reticulum but localizes to the extracellular surface. Glycosylation-variant BEHAB binds to the membrane by a calcium-independent mechanism that is distinct from glycosylated forms of BEHAB/brevican and other lecticans (Yamaguchi, 2000, Cell Mol. Life Sci. 57: 276-289), indicating that the underglycosylation of the protein directly affects its biochemical binding properties.

Glycosylation-Variant BEHAB Associates with Glioma Membranes by a Mechanism Distinct from Other BEHAB Isoforms

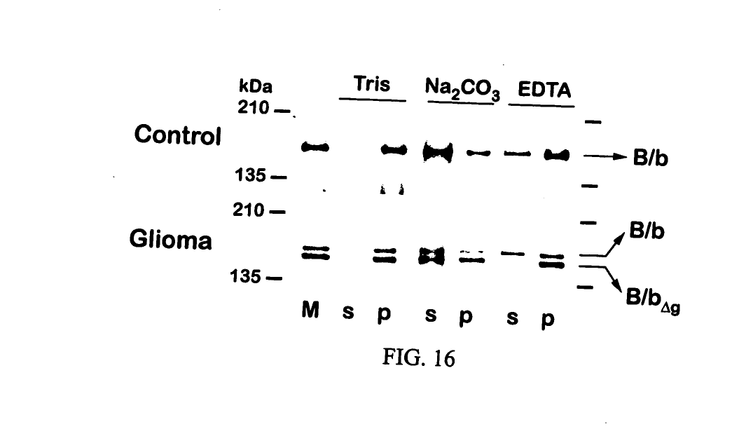

Glycosylation-variant BEHAB partitions with the particulate fraction of glioma and can reach the cell surface, leading to an investigation of whether the mechanism of membrane association was the same for full-length BEHAB and glycosylation-variant BEHAB was explored. First, membranes from normal brain and glioma were treated with sodium carbonate, which releases peripherally associated proteins, but does not release covalently-linked or integral membrane proteins. Both glycosylated full-length BEHAB and glycosylation-variant BEHAB were released in the same proportion into the soluble fraction (FIG. 16), confirming that glycosylation-variant BEHAB associates peripherally with the cell surface.

Since all known cell surface ligands of BEHAB associate with its lectin-like domain by a calcium-dependent mechanism (Asperg et al., 1997, Proc. Nat'l. Acad. Sci. USA 94: 10116-10121; Miura et al., 1999, J. Biol. Chem. 274: 11431-11438), membranes from normal brain and glioma were treated with EDTA to disrupt binding. EDTA treatment partially released the ˜160-kDa full-length BEHAB isoform in normal tissue and in glioma (FIG. 16), while glycosylation-variant BEHAB was not affected, indicating that it associates with the cell membranes by a unique, calcium-independent mechanism.

A BEHAB/brevican isoform in the rat brain with similar characteristics to human glycosylation-variant BEHAB but different expression pattern in normal brain has been described (Viapiano et al., 2003, J. Biol. Chem. 278: 33239-33247). Rat glycosylation-variant BEHAB is also the major upregulated form of BEHAB in rat experimental gliomas. The upregulation of the underglycosylated isoforms of BEHAB in both rat and human glioma suggests that regulated glycosylation of BEHAB can play a significant role in the progression of glial tumors. Indeed, glycosylation of the lecticans is precisely regulated in the CNS (Matthews et al., 2002, J. Neurosci. 22: 7536-7547). Furthermore, many of the functional properties lecticans in the CNS are in fact mediated by their attached carbohydrates (Bandtlow and Zimmerman, 2000, Physiol. Rev. 80: 1267-1290; Properzi and Fawcett, 2004, News Physiol. Sci. 19: 33-38). Therefore, lack of glycosylation in glycosylation-variant BEHAB can produce a molecule with very unique functional properties. CD44H, another key organizer of the neural ECM, is aberrantly under-glycosylated in neuroblastoma and binds defectively to the extracellular HA scaffold (Gross et al., 2001, Med. Pediatr. Oncol. 36: 139-141). The overexpression of glycosylation-variant BEHAB on the surface of glioma cells could therefore promote tumor progression by similarly disturbing the interactions of normal BEHAB and enabling novel cell-cell interactions that favor invasion.

Selective targeting of cancer cells through specific cell-surface antigens is an attractive therapeutic approach that is being currently explored for glioma (Kuan et al., 2001, Endocr. Relat. Cancer 8: 83-96; McLendon et al., 2000, J. Histochem. Cytochem. 48: 1103-1110; Leins et al., 2003, Cancer 98: 2430-2439). A major hurdle in this approach is the paucity of ideal molecular targets that are both restricted in expression to the tumor cells and are available at the cell surface. The selective expression of glycosylation-variant BEHAB in glioma, its restricted membrane localization, and its expression in all high-grade gliomas tested make it an important new target for therapy. In addition, the absence of glycosylation-variant BEHAB in a specific subset of low-grade, indolent oligodendrogliomas demonstrate its use as a diagnostic marker to distinguish primary brain tumors of similar histology but different pathological course. The clear clinical value of glycosylation-variant BEHAB, together with the role of BEHAB in promoting glioma progression demonstrate that this protein is a relevant candidate for novel anti-tumoral approaches in glioma.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated human poly-sialyated BEHAB polypeptide, wherein said poly-sialyated BEHAB polypeptide has a molecular weight greater than about 160 kDa and comprises the amino acid sequence set forth in SEQ ID NO: 8.

2. The isolated polypeptide of claim 1, wherein said molecular weight is from about 163 kDa to about 166 kDa.

3. The isolated polypeptide of claim 1, wherein said polypeptide comprises from about 10 to about 20 sialic acid residues more than full-length BEHAB.

4. The isolated polypeptide of 3, wherein said sialic acid residues are attached to said polypeptide via an O-linkage.

5. A method of detecting a malignant glioma in a mammal, said method comprising contacting a biological sample of said mammal with an antibody that specifically binds with a glycosylation-variant BEHAB polypeptide and detecting binding of said antibody to said biological sample, wherein binding of said antibody with said biological sample detects a malignant glioma in a mammal.

6. The method of claim 5, wherein said mammal is a human.

7. The method of claim 5, wherein said biological sample is a CNS tissue sample.

8. The method of claim 7, wherein said CNS tissue sample is a brain tissue.

9. The method of claim 5, wherein said antibody is selected from the group consisting of B5, B6, and B_{CRP}.

10. The method of claim 9, wherein said antibody comprises a tag covalently linked thereto.

11. The method of claim 5, wherein said glioma is a malignant high grade glioma.

12. A method of differentially diagnosing a malignant glioma from a benign glioma in a mammal, said method comprising contacting a biological sample of said mammal with an antibody that specifically binds with a glycosylation-variant BEHAB polypeptide and detecting binding of said antibody to said biological sample, wherein binding of said antibody with said biological sample detects a malignant glioma in a mammal.

13. The method of claim 12, wherein said mammal is a human.

14. The method of claim 12, wherein said biological sample is a CNS tissue sample.

15. The method of claim 14, wherein said CNS tissue sample is a brain tissue.

16. The method of claim 12, wherein said antibody is selected from the group consisting of B5, B6, and B_{CRP}.

17. The method of claim 16, wherein said antibody comprises a tag covalently linked thereto.

18. The method of claim 12, wherein said malignant glioma is a malignant high grade glioma.

19. The method of claim 12, wherein said benign glioma is a benign low grade glioma.

20. The method of claim 19, wherein said benign low grade glioma is a grade II glioma.

21. The method of claim 19, wherein said benign low grade glioma is an oligodendroglioma associated with chronic epilepsy.

22. A method of assessing a change in tumor progression in a mammal, said method comprising contacting a first biological sample of said mammal with an antibody that specifically binds with a glycosylation-variant BEHAB polypeptide and detecting binding of said antibody to said biological sample, said method further comprising comparing the level of glycosylation-variant BEHAB in said biological sample with the level of glycosylation-variant BEHAB in a second biological sample from said mammal, wherein a difference in the level of glycosylation-variant BEHAB in said first biological sample compared to the level of glycosylation-variant BEHAB in said second biological sample indicates a change in tumor progression in said mammal.

23. The method of claim 22, wherein said mammal is a human.

24. The method of claim 22, wherein said biological sample is a CNS tissue sample.

25. The method of claim 24, wherein said CNS tissue sample is a brain tissue.

26. The method of claim 22, wherein said antibody is selected from the group consisting of B5, B6, and B_{CRP}.

27. The method of claim 22, wherein said antibody comprises a tag covalently linked thereto.

28. The method of claim 22, wherein said glioma is a malignant high grade glioma

29. A kit for detecting a malignant glioma, said kit comprising an antibody that specifically binds with a glycosylation-variant BEHAB, said kit further comprising an applicator, and an instructional material for use thereof.

30. A kit for differentially diagnosing a malignant glioma from a benign glioma, said kit comprising an antibody that specifically binds with a glycosylation-variant BEHAB, said kit further comprising an applicator, and an instructional material for use thereof.

31. A kit for assessing a change in tumor progression in a mammal, said kit comprising an antibody that specifically binds with a glycosylation-variant BEHAB, said kit further comprising an applicator, and an instructional material for use thereof.
